SEQUENCE LISTING (2 sequences)

SEQ ID NO: 1 - protein, 20 aa, Artificial Sequence
  guide RNA (gRNA) to knock-in the specific point-mutation of interest
GGACATTGTTGACGTAAGGG

SEQ ID NO: 2 - DNA, 110 bp, Artificial Sequence
  single-stranded oligo donor (ssODN) template to knock-in the specific point-mutation of interest
ggcactgagcacacaaccaatgatgttcccaaggtgggggacattgttgacgtaagggccggcactggtgatgagcacattcctttctccagccacaggcaacctagtaa

(19) United States
(12) Patent Application Publication
(10) Pub. No.: US 2024/0117431 A1
(43) Pub. Date: Apr. 11, 2024

(54) STEM-CELL BASED BIOMARKER DISCOVERY

(71) Applicant: THE REGENTS OF THE UNIVERSITY OF CALIFORNIA, Oakland, CA (US)

(72) Inventors: Mathew Blurton-Jones, Irvine, CA (US); Jean Paul Chadarevian, Irvine, CA (US); Robert Spitale, Irvine, CA (US); Sunil Gandhi, Irvine, CA (US); Kim Nguyen, Irvine, CA (US)

(21) Appl. No.: 18/000,564

(22) PCT Filed: Jun. 2, 2021
(86) PCT No.: PCT/US21/35454

§ 371 Date: Dec. 2, 2022

Related U.S. Application Data
(60) Provisional application No. 63/033,508, filed on Jun. 2, 2020.

(51) Int. Cl. C12Q 1/6883; A01K 67/0271; C12N 5/079; C12N 9/00; C12N 9/12; C12Q 1/6806

(52) U.S. Cl. CPC C12Q 1/6883; A01K 67/0271; C12N 5/0618; C12N 9/12; C12N 9/93; C12Q 1/6806; A01K 2267/0312; C12Y 601/0101

(57) ABSTRACT

The present invention relates to the development of and use of genetically modified human differentiated cells coupled with xenotransplantation into animal models to identify injury and disease-specific RNA and/or protein biomarkers. Specifically, the present invention encompasses two complementary methods for biomarker discovery that enable the direct and selective labelling, isolation, and analysis of human-specific RNA and/or proteins from xenotransplantation (or chimeric) animal models. Both methods involve the treatment of animal models with an RNA analog and/or amino acid analog that enables the specific isolation and quantification of human RNAs and/or proteins for the identification of novel human biomarkers for a large array of human injuries and diseases.

### CROSS-REFERENCES TO RELATED APPLICATIONS

This application claims benefit of U.S. Provisional Application No. 63/033,508 filed Jun. 2, 2020, the specification of which is incorporated herein in their entirety by reference

### STATEMENT REGARDING FEDERALLY SPONSORED RESEARCH OR DEVELOPMENT

This invention was made with government support under Grant Nos. 1 RF1 DA048813-01 awarded by NIH BRAIN Initiative/NIDA, 5R21MH113062-02 awarded by NIMH, and R01 AG048099 awarded by NIA. The government has certain rights in the invention.

### REFERENCE TO A SEQUENCE LISTING

Applicant asserts that the information recorded in the form of an Annex C/ST.25 text file submitted under Rule 13ter.1(a), entitled UCI_20_10_PCT_Sequencing_Listing_ST25, is identical to that forming part of the international application as filed. The content of the sequence listing is incorporated herein by reference in its entirety.

### BACKGROUND OF THE INVENTION

The present invention addresses a critical need to identify new biomarkers that can predict and/or track the onset and progression of human diseases. Animal models do not necessarily provide an optimal tool to identify human biomarkers of injury or disease that may be species-specific. Profiling either RNA or protein expression in a cell-specific manner continues to be a grand challenge in biochemical research. Bioorthogonal nucleosides can be utilized to track RNA expression; however, these methods currently lack the high stringency for in vivo applications. Described herein, the present invention in part demonstrates that uracil phosphoribosyltransferase (UPRT) can be engineered to match 5-vinyluracil (5VU) for cell-specific metabolic labeling of human RNA, with exceptional specificity and stringency. The present invention further demonstrates the use of biorthogonal amino acid labeling to detect and identify human proteins derived from specific human cell populations. By combining these RNA and protein labeling approaches with xenotransplantation of human cells into appropriate animal models, this invention provides a new cell origin-specific approach to detect human RNA and protein biomarkers.

### FIELD OF THE INVENTION

The present invention relates to the development of and use of genetically modified human differentiated cells coupled with xenotransplantation into animal models to identify injury and disease-specific protein and RNA biomarkers. In particular, the present invention features complementary methods for biomarker discovery using selective, cell-specific labeling of human RNAs and proteins within chimeric xenotransplantation models of disease.

Specifically, the present invention encompasses two complementary methods that enable the direct labeling, isolation, and analysis of human-specific RNA and/or proteins from xenotransplantation (or chimeric) animal models. Both methods involve the genetic modification of human pluripotent stem cells including embryonic (ESCs), induced pluripotent stem cells (iPSCs), or derivatives thereof and their subsequent differentiation into a relevant cell type and xenotransplantation into a relevant animal model. Subsequent treatment of animals with a modified amino acid analog or RNA analog will enable direct labeling and specific isolation and quantification of human proteins and RNAs to identify novel human biomarkers for a large array of human injuries and diseases.

### BACKGROUND ART

The prior art related to this work includes the more standard approach of examining animal models of human diseases and using changes in animal RNA or proteins to try to predict changes that may or may not be detected in human patients. While this has identified some promising biomarkers for certain diseases, it has also led to a large number of candidate biomarkers that have failed to translate to the human condition.

The initial development and use of amino acid analogs to label proteins from specific murine cell populations has been previously described; the development of cell-type-specific protein labeling for examining protein expression from specific mouse cell types is described in US20060216760, “Methods for proteomic profiling using non-natural amino acids”. This publication describes the use of a methionine analog, L-Azidonorleucine (ANL) coupled with a mutant transfer RNA to label nascent proteins. ANL does not fit into the wild-type binding pocket of Methionine-tRNA Synthase (MetRS). However, introduction of a point mutation into Methionine-tRNA can enable the incorporation of this analog into nascent proteins. This approach was used to develop new mouse models that allow researchers to label proteins from a specific murine cell type. Others have used this approach in cell lines using multiple Click-Selective tRNA Synthetases to expand mammalian cell-specific proteomics.

WO2018160496 is related to the development of methods to differentiate pluripotent stem cells into hematopoietic progenitors and microglia. Importantly, this prior art does not include the use of the described methods of the present invention to enable species-specific protein or RNA labeling within chimeric models.

A powerful step in the development of chemical methods for tracking RNA expression is to make such approaches cell-specific, whereby RNAs of desired cells can be marked with specific chemical handles for analysis. The inventors have recently expanded the chemical methods for cell-specific metabolic labeling of RNA, by chemical diversifying nucleobases to make them become either activated by enzymes or de-“caged” such that liberated nucleobases can be eventually incorporated into cellular RNA. However, each of these has its own limitations.

“Caged” nucleobases are often protected by carbonyl groups which may be susceptible to hydrolysis and may not be stable enough to work in vivo. As an alternative approach, utilizing enzymes to convert inert metabolic intermediates into active ones has high merit as such intermediates are stable and can survive long incubation times in cells and in vivo.

For example, the present invention expands the substrate scope of uracil using Toxoplasma gondii uracil phosphoribosyltransferase (TgUPRT) with uracil analogs to produce modified 5′-phosphorylated uridines (FIG. 7A). As such, the UPRT system would be ideal because perfusion of small nucleobases throughout the entirety of an animal is expected to be robust and has been demonstrated with other modified uracil analogs (e.g., 4-thiouracil and toxic 5-fluorouracil). However, the previously used analogs do not possess the binary stringency (no incorporation when TgUPRT is not expressed; incorporated only when TgUPRT is expressed) that is desired for in vivo applications.

It has been recently observed that mammalian cells are able to salvage uracil for eventual incorporation into cellular RNA, without the expression of TgUPRT. As such, the cellular specificity of this approach is still limiting; therefore, limiting its utility in complex multicellular systems. To minimize the problem with background, a two-step enzymatic approach, which takes advantage of pairing 5-ethynylcytosine with a cytidine deaminase—UPRT pair was developed. However, this system would require longer exposure of the analogs to achieve desired RNA labeling, and the larger genetic size of a two-enzyme system limits its application for in vivo approaches. These major challenges leave critical components to be optimized but present a unique opportunity to expand the chemical repertoire of analogs to achieve high-stringency cell-specific RNA labeling. The present invention features a system matured for very robust and high stringency cell-specific metabolic labeling of RNA in a living animal.

The present invention moves well beyond these initial concepts by combining this molecular approach with human stem cells and xenotransplantation into animal models of human diseases. This chimeric animal labeling approach described herein has high clinical relevance that was not disclosed in prior studies. Importantly, US20060216760 does not mention or discuss human cells, human stem cells, transplantation, xenotransplantation, chimeric models, or biomarkers.

### BRIEF SUMMARY OF THE INVENTION

It is an objective of the present invention to provide methods that allow for human biomarker discovery using human differentiated cells, high-stringency cell-specific RNA and/or amino acid labeling, and xenotransplantation into animal models of human disease, injury, and/or pathology as specified in the independent claims. Embodiments of the invention are given in the dependent claims. Embodiments of the present invention can be freely combined with each other if they are not mutually exclusive.

One of the unique and inventive technical features of the present invention is the use of complementary methods of metabolic labeling and xenotransplantation. Without wishing to limit the invention to any theory or mechanism, it is believed that the technical feature of the present invention advantageously provides for the direct labeling, isolation, and analysis of human-specific RNA or proteins from xenotransplantation (or chimeric) animal models for human biomarker discovery. None of the presently known prior references or work has the unique inventive technical feature of the present invention. Furthermore, the prior references teach away from the present invention. For example, traditional (non-xenotransplanted) animal models do not necessarily provide an optimal tool to identify human biomarkers of injury or disease that may be species-specific.

The present invention addresses a critical need to identify new biomarkers that can predict and/or track the onset and progression of human diseases. The present invention features two complementary systems that enable the direct labeling, isolation, and analysis of human-specific RNA or proteins from xenotransplantation (or chimeric) animal models. Both systems involve the genetic modification of human pluripotent stem cells including embryonic (ESCs), induced pluripotent stem cells (iPSCs), or their derivatives thereof, their subsequent differentiation into a relevant cell type, and xenotransplantation into a relevant animal model. Subsequent treatment of animals with a specific amino acid analog or RNA analog will enable the specific isolation and quantification of human proteins and RNAs to identify novel human biomarkers for a large array of human injuries and diseases. A non-limiting example of a derivative of a stem cell comprises iPSCs differentiated into neural stem cells, and these iPSCs differentiated-neural stem cells can then be genetically modified.

The present invention features methods for cell-specific RNA and/or protein labeling. In some embodiments, the method comprise xenotransplanting genetically modified differentiated cells into an area of interest in an animal model. In some embodiments, the method comprises treating the animal of the model with an RNA (i.e., a uracil analog) and/or an amino acid analog. In some embodiments, the method comprises extracting total RNA and/or total protein from the area of interest in the animal model. In other embodiments, the method comprises producing a fraction of the RNA and/or protein extracted from the area of interest in the animal model. In some embodiments, a fraction of RNA and/or protein is produced by attaching a label to the RNA and/or the protein comprising the RNA analog (i.e., a uracil analog) and/or the amino acid analog and isolating the labeled RNA and/or the labeled protein from the total RNA and/or the total protein extracted from the area of interest in the animal model. In other embodiments, the method comprises analyzing the labeled RNA and/or the labeled protein isolated.

The present invention features methods for a stem cell-based approach for biomarker discovery. In preferred embodiments, this approach comprises first a genetic modification of human pluripotent stem cells. Non-limiting examples of these stem cells comprise ESCs, iPSCs, or derivatives thereof. Second, the approach comprises subsequent differentiation of ESCs, iPSCs, or derivatives thereof into a relevant cell type (for example, neural stem cells). The approach comprises subsequent xenotransplantation into a relevant animal model (e.g., model of human injury or disease, spinal cord injury model) and subsequent treatment of animals of the specific animal model with a modified amino acid analog and/or an RNA analog. The treatment will enable the specific isolation and quantification of human proteins and RNAs from the animals to identify human biomarkers for human injury and/or disease.

The present invention features that human stem cells can be genetically modified to introduce a mutation into human Methionyl-tRNA synthetase (MetRS). Using CRISPR gene editing, this locus was successfully modified in a human iPSC line (ADRC 76-generated by the UCI ADRC iPS cell core). In vitro validation subsequently confirmed that human nascent proteins can be specifically labeled with azidonorleucine (ANL) and isolated via Click chemistry for subsequent downstream analysis such as proteomics. By coupling this human iPSC line or similarly-modified stem cell lines with xenotransplantation, the present invention specifically labels human cells within a chimeric animal model. The present invention features similar modifications of other amino acid tRNA synthases that can also be used for this approach including modification of phenylalanine tRNA synthetase and tyrosine tRNA synthetase to allow biorthogonal labeling with labeled noncanonical amino acids.

The present invention builds upon the utility of a wild-type UPRT enzyme and pairing with modified nucleobase analogs for unique, cell-specific RNA labeling. The present invention features a novel mutant UPRT enzyme-modified analog (5-vinyluracil; 5VU), which has a higher specificity index for cell-specific RNA labeling. This novel metabolic labeling method can be employed to identify RNAs that come from a cell of interest (e.g., human iPSC lines). When RNAs in such cells are labeled, they can be isolated and purified.

Any feature or combination of features described herein are included within the scope of the present invention provided that the features included in any such combination are not mutually inconsistent as will be apparent from the context, this specification, and the knowledge of one of ordinary skill in the art. Additional advantages and aspects of the present invention are apparent in the following detailed description and claims.

### BRIEF DESCRIPTION OF THE SEVERAL VIEWS OF THE DRAWING(S)

The features and advantages of the present invention will become apparent from a consideration of the following detailed description presented in connection with the accompanying drawings in which:

FIG. 1A shows a chromatogram of the generated iPSC line. FIG. 1B shows western blot analysis of Wild-type (WT) and MethL272G iPSCs.

FIG. 2A shows confocal microscopy of ANL incorporation in iPSCs. FIG. 2B shows confocal microscopy of microglia derived from the Methionine-tRNA Synthetase L272G (MethL272G) iPSC line and cultured in the presence and absence of lipopolysaccharide (LPS) to induce changes in microglial protein expression.

FIG. 3A shows an assessment of background level of 5EU incorporation into RNA in (−)TgUPRT HEK cells with treatment of 5EUracil or 5EUridine (served as position control) at two different concentrations (0.2 mM and 1 mM) in a time course. Strep-HRP=Streptavidin conjugated Horseradish peroxidase. MeBI=methylene blue staining serving as loading control. Both analogs were incubated for 24 hours at 1 mM concentration in HEK cells.

FIG. 3B shows a dot blot demonstrating background incorporation of 4-thiouracil in comparison to 4-thiouridine in HEK, (−)TgUPRT, cells. RNA was extracted and biotinylated using MTSEA-Biotin, followed by dot-blot analysis. Strep-HRP=Streptavidin conjugated Horseradish peroxidase. MeBI=methylene blue (MB) staining serving as loading control.

FIG. 4 shows an assessment of four different uracil analogs incorporated into RNA without or with TgUPRT-WT or mutants by dot blot analysis. 2 μg biotinylated RNA was loaded per spot. Structures of different uracil analogs are provided in the left panel and the right panel shows images of the same blot at exposed time (top) when signal is still in linear range, (middle) when signal reaches saturation, (bottom) loading control. Strep-HRP=Streptavidin conjugated Horseradish peroxidase. MeBI=methylene blue staining serving as loading control. HEK cells are non-transfected cells.

FIGS. 5A, 5B, 5C, 5D, and 5E show confocal microscopy analysis of RNA labeling using 5VU and HEK containing TgUPRT variants. FIG. 5A shows triple mutant 3×MT-TgUPRT+1 mM 5VU-24h, FIG. 5B shows triple mutant 3×MT-TgUPRT+DMSO-24h, FIG. 5C shows WT-TgUPRT+1 mM 5VU-24h, FIG. 5D shows WT-TgUPRT+DMSO-24h, and FIG. 5E shows (−)TgUPRT HEK+1 mM 5VU-24h. Imaging was done via fluorescence confocal microscopy using a 63× oil immersion objective on a Leica 700 Carl Zeiss microscope. Hoechst for staining nuclei, Tz-Biotin=tetrazine-biotin, Strep-A488=Streptavidin-Alexa488. Merge=superimpose Hoechst staining and Strep-A488 images.

FIGS. 6A, 6B, and 6C show specificity assessment of the 2 (5VU) and GFP-3×MT-TgUPRT pair in co-culture and dot blot analysis. FIG. 6A shows a schematic of a co-culture experiment. FIG. 6B shows a dot blot analysis of total RNA isolated from co-culture of HEK cells containing mCherry plasmid and HEK cells transfected with GFP-WT-TgUPRT or GFP-3×MT-TgUPRT plasmid. The co-culture HEK cells were treated with both 200 uM 5EU and 500 uM 5VU for 5h. FIG. 6C shows microscopy analysis of the co-culture prior treatment of dual 5EU-5VU analogs.

FIGS. 7A, 7B, and 7C show UPRT-dependent metabolic labeling of RNA. FIG. 7A shows. Schematic of (−) TgUPRT expressing cell versus TgUPRT expressing cells that enable cell-specific metabolic labeling of RNA. FIG. 7B. shows a crystal structure of Toxoplasma gondii UPRT enzyme (PDB 1bd4). FIG. 7C shows a close-up view of Toxoplasma gondii UPRT active site. Positions chosen for mutagenesis are labeled.

FIGS. 8A, 8B, 8C, and 8D show in-cell screening of TgUPRT mutants matched with bioorthogonal analogs. FIG. 8A shows chemical structures of uracil and uracil analogs used herein. FIG. 8B shows schematic of in-cell screening experiments. HEK293 cells were transfected with TgUPRT plasmids containing various mutations. Uracil analogs were added at 200 μM and incubated for 5 hours. Following RNA isolation, biotinylation was performed and incorporation of analog was determined by streptavidin dot blot. FIG. 8C shows dot blot screening for RNA incorporation of four different uracil analogs by fifteen TgUPRT mutants. FIG. 8D shows higher exposure of the dot blot represented in FIG. 80.

FIGS. 9A and 9B show in vitro analysis of activity of TgUPRT mutants for phosphoribosyl-transferase activity of different uracil analogs. FIG. 9A shows specific activity of TgUPRT variants with different uracil analogs (1 to 4 as shown in FIG. 8A). n=3 technical replicates. FIG. 9B shows time course analysis of uracil analog incorporation into RNA by streptavidin dot blot (left panels) along with western blot analysis (right panels) of corresponding protein levels of 6×His-TgUPRT. α-His=anti-his antibody for western blot. Strep-HRP=Streptavidin conjugated Horseradish peroxidase was used for assessment of biotin level resulting from clicked RNA. MeBI=Methylene blue stain and CS=coomassie staining served as loading control of RNA and proteins, respectively.

FIGS. 10A, 10B, 100, 10D, 10E show characterizing the stringency of the 2 (5VU)-mutant TgUPRT pair. FIG. 10A shows microscopy analysis RNA incorporation of 2 in a mutant TgUPRT-dependent manner. Cells were transfected with TgUPRT variants and incubated with 2 at 1 mM final concentration for 24h. 2 incorporation was imaged using two-step labeling (1) IEDDA using tetrazene-biotin then followed by Alexa488-streptavidin. FIG. 10B shows a schematic of co-culture experiment to assess specificity 2/TgUPRT variants treated with both 200 μM5EU and 500 μM 5VU. FIG. 100 shows a presentation of mCherry-containing HEK cells co-cultured with TgUPRT-GFP variant transfected HEK cells. FIG. 10D shows a dot blot analysis of RNA isolated from co-cultured cells that underwent either CuAAC or IEDDA click. Streptavidin-HRP (Strep-HRP) was used for assessment of biotin level resulting from clicked RNA, and methylene blue (MeBI) staining served as loading control. FIG. 10E shows RT-qPCR enrichment of GFP transcripts and their fold enrichment over cell off-target mCherry transcript. Cells were treated the same as in panel 10B and the RNA appended with biotin and enriched before RT RT-qPCR.

FIG. 11 shows an assessment of cell proliferation under treatment of 5-vinyl uracil time course in the presence or absence of mutant TgUPRT (double=2×MT or triple=3×MT mutations). Equal amount HEK cells (2×10⁵) were seeded in a 6-well plate with 2 mL media. Twenty-four hour post transfection, HEK cells were transfected with 1 μg empty plasmid (No GFP nor TgUPRT), double (2×MT) or triple (3×MT) TgUPRT mutants. 5VU was added to cells at 200 μM final concentration for 5, 16 and 24h treatment so that by the time cells were assayed with Trypan blue, they underwent 48h post transfection. DMSO (carrier) treatment (negative control) was done for 24h. Cells were stained with 0.04% Trypan blue for 5 minutes and subjected to cell counting using Countess II FL (Invitrogen, USA). Per condition, counting was done with n=2× biological and 3× technical replicates.

FIG. 12 shows a dot blot demonstrating incorporation of 1 and 4-thiouridine, but not 2 (5VU) into cellular RNA with the overexpression of UMPS. Analogs were incubated for 24 hours at 1 mM concentration in HEK cells. RNA was extracted and biotinylated using MTSEA-Biotin, followed by dot-blot analysis. Strep-HRP=Streptavidin conjugated Horseradish peroxidase. MeBI=methylene blue staining serving as loading control.

FIGS. 13A, 13B, 13C, and 13D show spectra of RNA analogs.

FIG. 14 shows a dot blot analysis demonstrates incorporation of 5-vinyluridine into RNA with UPRT(+)-microglia transplanted. Biotin=biotinylation conjugation to RNA. Methylene blue=nucleic acid stain for measuring loading control.

### DETAILED DESCRIPTION OF THE INVENTION

ESC: embryonic stem cell
iPSC: induced pluripotent stem cell
ANL: L-azidonorleucine
5EU: 5-ethynylpyrimidine-2,4(11-1,3H)-dione
5VU: 5-Vinylpyrimidine-2,4(1H,3H)-dione
AMUra or 5mAzU: 5-azido methyl uracil
5AU or 5AzU: 5-Azidopyrimidine-2,4(1H,3H)-dione
FUNCAT: Functional Catalogue
BONCAT: Bioorthogonal non-canonical amino acid tagging

As used herein, the term “genetically-modified” refers to (of an organism or crop) containing genetic material that has been artificially altered so as to produce a desired characteristic.

As used herein, the term “stem cell” refers to in multicellular organisms, stem cells are undifferentiated or partially differentiated cells that can differentiate into various types of cells and divide indefinitely to produce more of the same stem cell. They are the earliest type of cell in a cell lineage. They are found in both embryonic and adult organisms, but they have slightly different properties in each. They are usually distinguished from progenitor cells, which cannot divide indefinitely, and precursor or blast cells, which are usually committed to differentiating into one cell type.

As used herein, the term “chimera” refers to an organism containing a mixture of genetically different tissues, formed by processes such as fusion of early embryos, grafting, or mutation.

As used herein, the term “chimeric transplant” refers to the placement (transplantation) of human cells into an animal model recipient.

As used herein, the term “biomarker” refers to bio-marker, or biological marker is a measurable indicator of some biological state or condition. Biomarkers are often measured and evaluated to examine normal biological processes, pathogenic processes, or pharmacologic responses to a therapeutic intervention. Biomarkers are used in many scientific fields and can be derived from any tissue or fluid including blood, urine, and cerebrospinal fluid.

As used herein, the term “Click-Chemistry” refers to a chemical synthesis, “click” chemistry is a class of biocompatible small molecule reactions commonly used in bioconjugation, allowing the joining of substrates of choice with specific biomolecules. Click chemistry is not a single specific reaction, but describes a way of generating products that follow examples in nature, which also generates substances by joining small modular units. In many applications, click reactions join a biomolecule and a reporter molecule. Click chemistry is not limited to biological conditions: the concept of a “click” reaction has been used in pharmacological and various biomimetic applications. However, they have been made notably useful in the detection, localization and qualification of biomolecules.

As used herein, the term “xenotranplantation” refers to the placement (transplantation) of cells from one species into a recipient organism of another species. For example, transplantation of human cells into a mouse.

As used herein, the term “biorthogonal” refers to bioorthogonal chemistry referring to any chemical reaction that can occur inside of living systems without interfering with native biochemical processes. The concept of the bioorthogonal reaction has enabled the study of biomolecules such as glycans, proteins, and lipids in real time in living systems without cellular toxicity. A number of chemical ligation strategies have been developed that fulfill the requirements of bioorthogonality, including the 1,3-dipolar cycloaddition between azides and cyclooctynes (also termed copper-free click chemistry), between nitrones and cyclooctynes, oxime/hydrazone formation from aldehydes and ketones, the tetrazine ligation, the isocyanide-based click reaction, and most recently, the quadricyclane ligation.

As used herein, the term “biorthogonal nucleoside” refers to a DNA or RNA molecule (nucleoside) that is chemically modified to allow specific detection and/or isolation.

As used herein, the term “proteome” refers to the entire set of proteins that is, or can be, expressed by a genome, cell, tissue, or organism at a certain time. It is the set of expressed proteins in a given type of cell or organism, at a given time, under defined conditions. Proteomics is the study of the proteome.

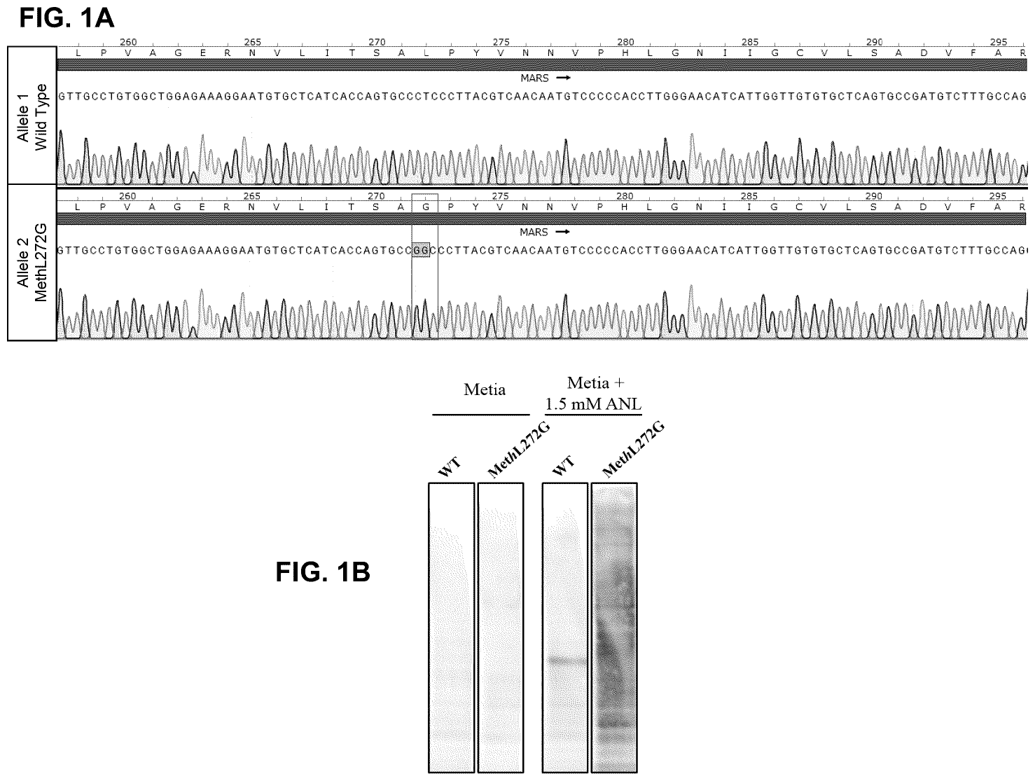

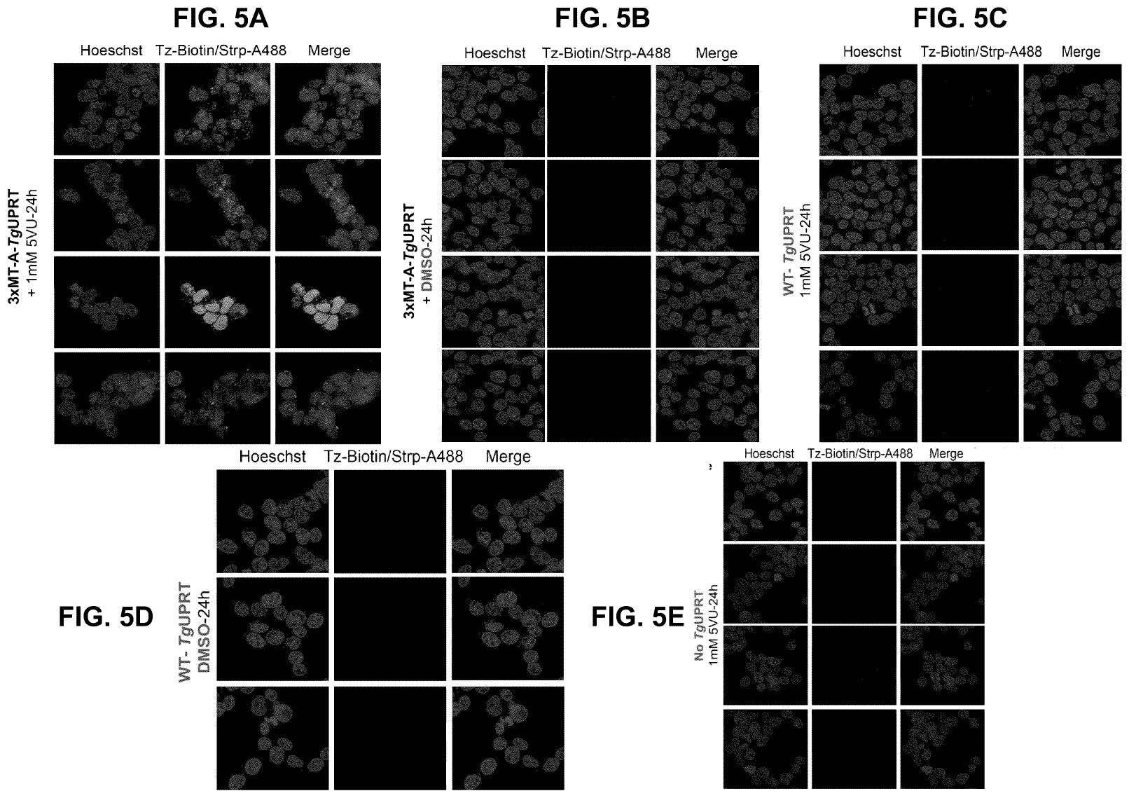

Referring now to FIGS. 1A-14, the present invention features methods for a cell-based approach for biomarker discovery. In preferred embodiments, the present invention features methods for cell-specific RNA and/or protein labeling. In some embodiments, the method comprises xenotransplanting genetically modified differentiated cells into an area of interest in an animal model. In some embodiments, the genetically modified differentiated cells comprise a genetically modified Uracil PhosphoRibosylTransferase (UPRT) enzyme, a genetically modified Methionine tRNA Synthase (MetRS) enzyme, or a combination thereof. In some embodiments, the method comprises treating the animal model with an RNA (i.e., a uracil analog) and/or an amino acid analog. In some embodiments, the method comprises extracting total RNA and/or total protein from the area of interest in the animal model. In other embodiments, the method comprises producing a fraction of the RNA and/or protein extracted from the area of interest in the animal model. In some embodiments the fraction of RNA and/or protein is produced by attaching a label to the RNA and/or the protein comprising the RNA analog (i.e., a uracil analog) and/or the amino acid analog and isolating the labeled RNA and/or the labeled protein from the total RNA and/or the total protein extracted from the area of interest in the animal model. In other embodiments, the method comprises analyzing the labeled RNA and/or the labeled protein isolated. In some embodiments, the method further comprises extracting total RNA and/or protein from the brain, plasma, cerebrospinal fluid (CSF), urine or a combination thereof from the animal model

The present invention may also feature a method for cell-specific RNA labeling. In some embodiments, the method comprises xenotransplanting genetically modified differentiated cells into an area of interest in an animal model. In other embodiments, the genetically modified differentiated cells comprise a genetically modified Uracil PhosphoRibosylTransferase (UPRT) enzyme. In some embodiments, the method comprises treating the animal of the model with an RNA analog (i.e., a uracil analog). In some embodiments, the method comprises extracting total RNA from the area of interest in the animal model. In other embodiments, the method comprises producing a fraction of the RNA extracted from the area of interest in the animal model. In some embodiments, a fraction of RNA is produced by attaching a label to the RNA comprising the RNA analog (i.e., a uracil analog) and isolating the labeled RNA from the total RNA extracted from the area of interest in the animal model. In some embodiments, the method comprises analyzing the labeled RNA isolated.

In some embodiments, the method further comprises extracting total RNA from the brain, the plasma, the cerebrospinal fluid (CSF), the urine or a combination thereof from the animal model. In some embodiments, the method further comprises producing a fraction of the RNA extracted from the brain of the animal model. In some embodiments, the method further comprises producing a fraction of the RNA extracted from the plasma of the animal model. In some embodiments, the method further comprises producing a fraction of the RNA extracted from the CSF of the animal model. In some embodiments, the method further comprises producing a fraction of the RNA extracted from the urine of the animal model. In some embodiments, the method further comprises producing a fraction of the RNA extracted from the brain, the plasma, the cerebrospinal fluid (CSF), the urine or a combination thereof from the animal model.

In some embodiments, the RNA analog is a uracil analog. In other embodiments, the RNA analog comprises uracil-based analogs. In some embodiments, the uracil-based analogs comprise 5-ethynylpyrimidine-2,4(1H,3H)-dione (5EU), 5-Vinylpyrimidine-2,4(1H,3H)-dione (5VU), 5-azido methyl uracil (5mAzU), 5-Azidopyrimidine-2,4(1H,3H)-dione (5AzU), or a combination thereof.

In some embodiments, the methods described herein are for cell-specific RNA labeling and uses labeling of 5EU, 5VU, 5mAzU, and 5AzU. Non-limiting examples of the RNA analogs comprise uracil-based analogs selected from the group consisting of 5EU, 5VU, 5mAzU, and 5AzU. In other embodiments, the RNA labelling is dependent on uracil phosphoribosyltransferase (UPRT).

In some embodiments, the genetically modified UPRT enzyme is a genetically modified Toxoplasma gondii Uracil PhosphoRibosylTransferase (TgUPRT) enzyme. In some embodiments, the Uracil PhosphoRibosylTransferase (UPRT) enzyme comprises point mutations. In some embodiments, the point mutations comprise M166A, A168G, Y228A, or a combination thereof.

The present invention may further comprise a method for cell-specific protein labeling. In some embodiments, the method comprises xenotransplanting genetically modified differentiated cells into an area of interest in an animal model. In some embodiments, the differentiated cells comprise a genetically modified Methionine tRNA Synthase (MetRS) enzyme. In some embodiments, the method comprises treating the animal of the model with an amino acid analog. In some embodiments, the method comprises extracting total protein from the area of interest in the animal model. In some embodiments, the method comprises producing a fraction of the protein extracted from the area of interest in the animal model. In other embodiments, a fraction of protein is produced by attaching a label to the protein comprising the amino acid analog and isolating the labeled protein from the total protein extracted from the area of interest in the animal model. In some embodiments, the method comprises analyzing the aforementioned labeled protein isolated.

In some embodiments, the method further comprises extracting total protein from the brain, the plasma, the cerebrospinal fluid (CSF), the urine or a combination thereof from the animal model. In some embodiments, the method further comprises producing a fraction of the protein extracted from the brain of the animal model. In some embodiments, the method further comprises producing a fraction of the protein extracted from the plasma of the animal model. In some embodiments, the method further comprises producing a fraction of the protein extracted from the CSF of the animal model. In some embodiments, the method further comprises producing a fraction of the protein extracted from the urine of the animal model. In some embodiments, the method further comprises producing a fraction of the protein extracted from the brain, the plasma, the cerebrospinal fluid (CSF), the urine or a combination thereof from the animal model.

In some embodiments, the amino acid analogs comprise azidonorleucine (ANL), azidotyrosine, azidophenylalanine, or a combination thereof. In some embodiments, the Methionine tRNA Synthase (MetRS) enzyme comprises point mutations. In other embodiments, the Methionine tRNA Synthase (MetRS) enzyme comprises a point mutation. In some embodiments, the point mutation comprises a L272G (MethL272G).

In some embodiments, the methods described herein are for cell-specific protein labeling and uses labeling of azidonorleucine, azidotyrosine, and/or azidophenylalanine. Non-limiting examples of amino acid analogs comprise azidonorleucine (ANL), azidotyrosine, and/or azidophenylalanine. In other embodiments, the protein labelling is dependent on a Methionine tRNA Synthase (MetRS) enzyme.

In some embodiments, the methods described herein further comprise differentiating stem cells. In some embodiments, the stem cells are differentiated before xenotransplantation into an area of interest in the animal model. In some embodiments, the stem cells are selected from a group consisting of embryonic (ESCs), induced pluripotent stem cells (iPSCs), neuronal stem cells, or derivatives thereof. In some embodiments, the differentiated cells are human differentiated cells. In other embodiments, the differentiated cells are selected from a group consisting of neurons, microglia, astrocytes, oligodendrocytes and other cells of the central nervous system.

As used herein, an “area of interest” refers to an area within an animal model. In some embodiments, an area of interest refers to an area within an animal model which is affected by a disease (e.g., the brain in an Alzheimer's Disease model animal). In other embodiments, an area of interest refers to an area in a control animal model which corresponds to the area affected by a disease in a disease animal model.

In some embodiments, the area of interest is the nervous system. In some embodiments, the area of interest is the brain. In some embodiments, the area of interest is the spinal cord. In some embodiments, the animal model is a chimeric animal model. In some embodiments, the animal model is an animal model of disease. In some embodiments, the animal model of disease is an animal model of Alzheimer's Disease. In some embodiments, the animal model of Alzheimer's Disease comprises beta-amyloid plaques. In other embodiments, the animal model of Alzheimer's Disease comprises neurofibrillary tangles. In some embodiments, the animal model of disease is an animal model of Parkinson's Disease. In some embodiments, the animal model of disease is an animal model of frontotemporal dementia. In some embodiments, the animal model is an animal model of injury. In some embodiments, the animal model of injury is a spinal cord injury animal model. In some embodiments, the animal model of injury is a traumatic brain injury animal model. In some embodiments, the animal model is a control animal model. In other embodiments, the control animal model has no disease and/or injury.

In preferred embodiments, the methods described herein are for studying human injury and/or disease. These methods comprise genetically-modified human stem cells or genetically-modified human differentiated cells coupled with xenotransplantation of the cells into an animal model representing human disease, injury, or pathology. These methods can then identify injury and human disease-specific protein and/or RNA biomarkers.

As used herein, an “animal model” refers to a living, non-human, often genetically-engineered animal used during the research and investigation of human disease or injury, for the purpose of better understanding the disease/injury process without the added risk of harming an actual human. In some embodiments, a control animal model (i.e., a control animal) refers to an animal model in which a certain disease or condition does not develop. In some embodiments, a control animal is used to validate a result. In some embodiments, a control animal model is a healthy or wild type (WT) animal model without disease or injury. The animal models described/used herein are well known in the art.

Non-limiting examples of animal models of human injury, disease, and/or pathology comprise models of Alzheimer's Disease, spinal cord injury, traumatic brain injury, Parkinson's disease, and/or frontotemporal dementia. Non-limiting examples of a specific or relevant or specific cell type comprise neuronal stem cell, neuronal cell, neurons, microglia, astrocytes, oligodendrocytes, and other cells of the central nervous system, embryonic stem cell, iPSC-differentiated cell, and/or a cell type specific to a human disease, or injury, or pathology.

In some embodiments, the fraction of labeled RNA changes in response to the beta-amyloid plaques within the Alzheimer's Disease animal model. In some embodiments, the fraction of labeled RNA changes in response to the neurofibrillary tangles within the Alzheimer's Disease animal model. In some embodiments, the fraction of labeled protein changes in response to the beta-amyloid plaques within the Alzheimer's Disease animal model. In some embodiments, the fraction of labeled protein changes in response to the neurofibrillary tangles within the Alzheimer's Disease animal model.

In some embodiments, the methods described herein are for human biomarker discovery and comprises genetically-modified human stem cells and xenotransplantation of the stem cells into a non-human animal model. In some embodiments, the method identifies injury and/or human disease-specific protein and RNA biomarkers.

In some embodiments, the methods described herein are for human biomarker discovery and comprises two complementary methods for the labeling of RNA (i.e., UPRT for labeling RNA) and/or protein (i.e, MetRS for labeling protein). In some embodiments, the two complementary methods enable direct labelling, isolation, and analysis of human-specific RNA and/or proteins from xenotransplantation (or chimeric) animal models.

In some embodiments, the method described herein are for biomarker discovery and comprise comparing labeled RNA isolated from the total RNA extracted from an animal model of disease to the labeled RNA isolated from total RNA extracted from a control animal model. In some embodiments, the method described herein are for biomarker discovery and comprise comparing labeled protein isolated from total protein extracted from an animal model of disease to labeled protein isolated from total protein extracted from a control animal model. In some embodiments, a biomarker is discovered when there is a change in the level of labeled RNA and/or the labeled protein in the animal model of disease compared to the control animal. In some embodiments, the level of labeled RNA and/or label protein in the animal model of disease decreases compared to the label RNA and/or labeled protein in the control animal model. In some embodiments, the level of labeled RNA and/or label protein in the animal model of disease increases compared to the label RNA and/or labeled protein in the control animal model.

In some embodiments, the method described herein are for biomarker discovery and comprise comparing labeled RNA isolated from the total RNA extracted from an animal model of injury to the labeled RNA isolated from total RNA extracted from a control animal model. In some embodiments, the method described herein are for biomarker discovery and comprise comparing labeled protein isolated from total protein extracted from an animal model of injury to labeled protein isolated from total protein extracted from a control animal model. In some embodiments, a biomarker is discovered when there is a change in the level of labeled RNA and/or the labeled protein in the animal model of injury compared to the control animal. In some embodiments, the level of labeled RNA and/or label protein in the animal model of injury decreases compared to the label RNA and/or labeled protein in the control animal model. In some embodiments, the level of labeled RNA and/or label protein in the animal model of injury increases compared to the label RNA and/or labeled protein in the control animal model.

In some embodiments, the methods described herein are for cell-specific RNA labeling. In other embodiments, the methods described herein are for cell-specific protein labeling. In further embodiments, the methods described herein are for cell-specific RNA labeling and cell-specific protein labeling. In some embodiments, the methods described herein provide for cell-specific biorthogonal metabolic labeling.

In some embodiments, the method described herein provides cell-specific biorthogonal metabolic labeling. The method described herein is used for human cells, human stem cells, transplantation, xenotransplantation, chimeric models, or biomarkers. In some embodiments, the method described herein comprises background RNA incorporation which does not result in enrichment of transcripts in off-target cells. In some embodiments, “background incorporation” may refer to metabolic incorporation of an RNA or protein analog into RNA or protein in cells not expressing a genetically modified UPRT enzyme or genetically modified MetRS. In other embodiments, “low background” may refer to metabolic incorporation of an analog below the detection limit of assays for measuring incorporation into cellular RNA or protein such as HPLC.

In some embodiments, the methods described herein comprise novel nucleobase-enzyme pairs for stringent, low RNA background incorporation and cell-specific metabolic labeling of RNA. A non-limiting example of the novel nucleobase-enzyme pair comprises a triple mutant (3×MT)-TgUPRT/2 (5-VU) pair to enrich RNAs specifically from target cells. In certain embodiments, the methods described herein are for in-cell screening for reactivity with mutant-analog pairs (structure 2/5VU and mutants TgUPRT) that are specific and have undetectable background activity with WT enzymes. In some embodiments the mutant analog pairs comprise C-5 modified uracil analogs compatible with triple mutants (3×MT), M166A/A168G/Y228A and M166A/A168G/Y228G, wherein the pairs enable robust incorporation of 5-vinyluracil (5VU).

In some embodiments, the methods described herein comprise novel amino acid-enzyme pairs for stringent, low protein background incorporation and cell-specific metabolic labeling of protein. In some embodiments the iPSC line comprises one wild-type copy and one mutated Methionine-tRNA Synthase L272G (MethL272G).

In select embodiments, the amino acid analog-incorporated human cell-derived proteins are isolated from brain, plasma, CSF, or urine from animals with human injury, disease, and/or pathology. For example, differences in levels of amino acid analog-incorporated human cell-derived proteins are detected in response to beta-amyloid Alzheimer's Disease pathology. Another non-limiting example of the present invention comprises a method that utilizes engineered MethL272G iPSC line to study the proteome of transplanted human microglia in Alzheimer's Disease mice.

In certain embodiments, the methods of the present invention are used for characterizing human cell-derived RNAs isolated from brain, plasma, CSF, or urine from animals with human injury, disease, and/or pathology. For example, differences in human RNA levels detected in response to beta-amyloid or neurofibrillary tangle Alzheimer's Disease pathology.

In some embodiments, the present invention comprises transplanting modified iPSC lines into models of human neuronal injury or disease comprising Alzheimer's disease, spinal cord injury, traumatic brain injury, Parkinson's disease, and/or frontotemporal dementia.

In some embodiments, the present invention features a chimeric microglial mouse model, but is not limited to studies of human microglia. In other embodiments, any human cell type can be produced from pluripotent stem cells and transplanted into an appropriate organ or location within a relevant animal model. By labeling and comparing human cell-derived proteins from control and affected animals, researchers will be able to identify novel biomarkers for an array of injuries and diseases. Subsequent validation of those candidate biomarkers within human subjects could then lead to the development of more specialized and specific assays for measuring these biomarkers such as ELISA analysis of CSF or plasma samples.

In some embodiments, the RNA is labeled with biotin. In some embodiments, the biotin is conjugated with an alkyne or azide. In other embodiments, the biotin is conjugated with tetrazene. In some embodiments, isolated RNA is appended with biotin using either copper-catalyzed azide-alkyne cycloaddition (CuAAC), with biotin conjugated alkyne or azide (for azido- and alkynyl-uracil analogs) or an inverse electron-demand Diels-Alder with a biotin-conjugated tetrazene (IEDDA). In further embodiments, biotinylation of RNA is assayed using streptavidin-HRP dot blot.

In some embodiments, the present invention features a method of cell-specific RNA labeling. In some embodiments, the method comprises xenotransplanting human pluripotent stem cell derived microglia (iMGL) into an animal model's brain. In some embodiments, the iMGL comprises a genetically modified Uracil PhosphoRibosylTransferase (UPRT) enzyme. In some embodiments, the method comprises treating the animal of the model with an uracil analog. In some embodiments, the method comprises extracting total RNA from the animal model's brain. In some embodiments, the method comprises producing a fraction of RNA. In some embodiments, a fraction of RNA is produced by attaching a label to the RNA comprising the uracil analog and isolating the labeled RNA from the total RNA extracted from the brain. In some embodiments, the method comprises analyzing the RNA isolated. In some embodiments, the method further comprises extracting total RNA from the CSF fluid, urine or a combination thereof from the animal model. In some embodiments, the uracil analog comprises 5-ethynylpyrimidine-2,4(1H,3H)-dione (5EU), 5-Vinylpyrimidine-2,4(1H,3H)-dione (5VU), 5-azido methyl uracil (5mAzU), 5-Azidopyrimidine-2,4(1H,3H)-dione (5AzU), or a combination thereof. In other embodiments, the genetically modified UPRT enzyme is a genetically modified Toxoplasma gondii Uracil PhosphoRibosylTransferase (TgUPRT) enzyme. In some embodiments, the genetically modified UPRT enzyme comprises point mutations. In further embodiments, the point mutations comprise M166A, A168G, Y228A, or a combination thereof.

In some embodiments, the method comprises: a) genetically modifying human pluripotent stem cells comprising ESCs, iPSCs, or derivatives thereof; b) differentiating of ESCs or iPSCs into a specific, differentiated type of cell; c) xenotransplanting the into a relevant animal model; and d) treating the animals of the relevant human disease animal model with an amino acid analog and/or an RNA analog enabling direct labelling and specific isolation and quantification of human proteins and/or RNAs to identify novel human biomarkers for human injury and/or disease.

### Example

The following are non-limiting examples of the present invention. It is to be understood that said examples are not intended to limit the present invention in any way. Equivalents or substitutes are within the scope of the present invention.

The development and in vitro validation has been completed for the first MethL272G iPSC line. Chromatogram and validation of ANL incorporation are shown in FIGS. 1A, 1B. Immunodeficient mice were transplanted with human hematopoietic progenitors (HPCs) derived from this iPSC line to produce chimeric mice in which human microglia have widely engrafted into the forebrain. The pairing of specific mutants with unique nucleoside analogs has been determined for cell-specific metabolic labeling of RNA as described below.

Labeling of nascent human proteins: A novel iPSC line was developed with one wild-type copy and one mutated Methionine-tRNA Synthetase L272G (MethL272G). This was done utilizing CRISPR-methods (as explained below) with the following guide RNA (gRNA) and single-stranded oligo donor (ssODN) template to knock-in the specific point-mutation of interest: gRNA: 5′ GGACATTGTTGACGTAAGGG (SEQ ID NO: 1) and ssODN template: 5′ ggcactgagcacacaaccaatgatgttcccaaggtgggggacattgttgacgtaagggccggcactggtgatgagcacattcctttctccagccaca ggcaacctagtaa 3′ (SEQ ID NO: 2)

CRISPR-Methods: 2×105 cells were isolated and single-celled using TrypLE Express (Gibco 12605028) enzymatic digestion for 3 min at 37C. Cells were resuspended in 60 μl nucleofection buffer from Human Stem Cell Nucleofector™ Kit 2 (Lonza VPH-5022). The suspension was combined with 2 uM ssODN template and 50 μg of RNP complex formed by incubating Alt-R® S.p. HiFi Cas9 Nuclease V3 (IDTDNA 1081061) with fused crRNA:tracrRNA (IDTDNA 1072534) duplex for 15 min at 23° C. The suspension was transferred to the Amaxa Nucleofector cuvette and transfected using program B-016. Cells were plated in TeSR™-E8™ (STEMCELL Technologies 05990) media with 0.25 μM Thiazovivin (STEMCELL Technologies 72252) overnight to recover. Cells were digested the following day with Accutase and single-cell plated to 96-well plates in TeSR™-E8™ media with 0.25 μM Thiazovivin and CloneR™ (STEMCELL Technologies 05888) supplement for clonal isolation and expansion. Genomic DNA was extracted using Extracta DNA prep for PCR (Quantabio 95091) from a sample of each clone upon passage and amplified for sequencing using Taq PCR Master Mix (ThermoFisher Scientific K0172) at the target site. PCR product from promising clones was transformed using TOPO™ TA Cloning™ Kit for Subcloning, with One Shot™ TOP10 (ThermoFisher Scientific K450040) for allele-specific sequencing.

Validation by Western Blot: Wild-type (WT) and MethL272G iPSCs were cultured in 6-well plate with and without 1.5 mM ANL (Tocris 6585) for 14 hrs in DMEM/F12 Silac media without Methionine (AthenaES 0433) supplemented with TeSR™-E8™ 25× Supplement. (Note: Methionine-absent media is described as Metia below.) Cells were dissociated and collected using TrypLE Express (Gibco 12605028) for 3 min at 37° C., isolated, and lysed in Ripa buffer with Halt Protease Inhibitor Cocktail (ThermoFisher Scientific 78429) and Halt Phosphatase Inhibitor Cocktail (ThermoFisher Scientific 78426). 200 μg of protein was labeled for analysis using Click-iT Protein Analysis TAMRA alkyne Detection Kit (Invitrogen C33370) that tags the azide of ANL incorporated protein with a TAMRA-alkyne tag via the following reaction:

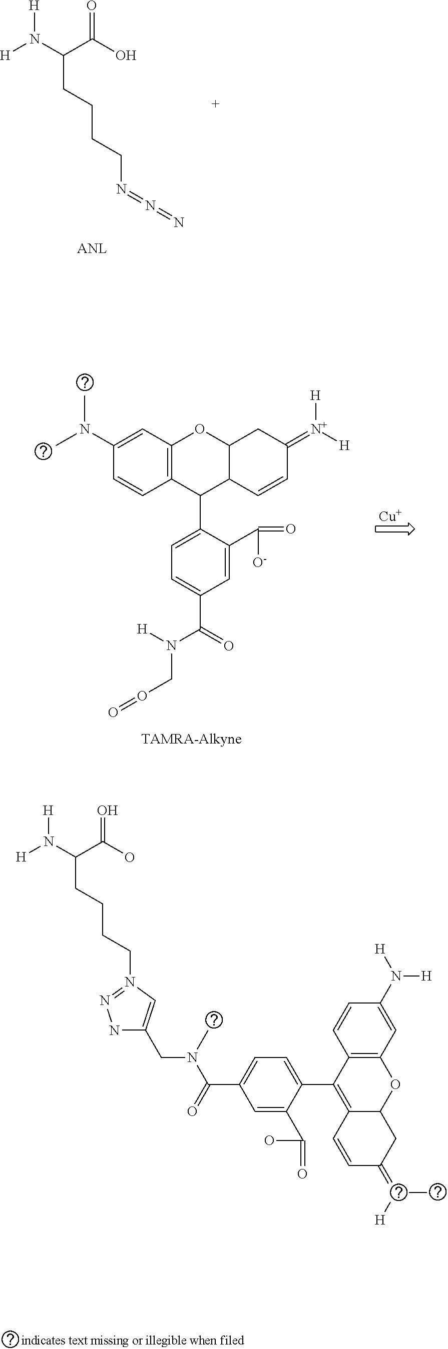

The resulting protein pellets were boiled in 120 μl of Loading Buffer and boiled for 15 minutes at 70° C., which were then loaded into a 4-15% Criterion TGX 12-well Gel (Bio-Rad 5671083) for electrophoresis, then transferred to Trans-Blot® Turbo™ Midi Nitrocellulose Membrane (Bio-Rad 1704159) for analysis. Protein was probed with TAMRA Monoclonal Antibody (5G5) (Invitrogen MA1-041) followed by anti-mouse secondary for imaging. FIG. 1B demonstrates the successful BONCAT labeling of ANL-incorporated protein isolated from ANL-incubated MethL272G iPSCs.

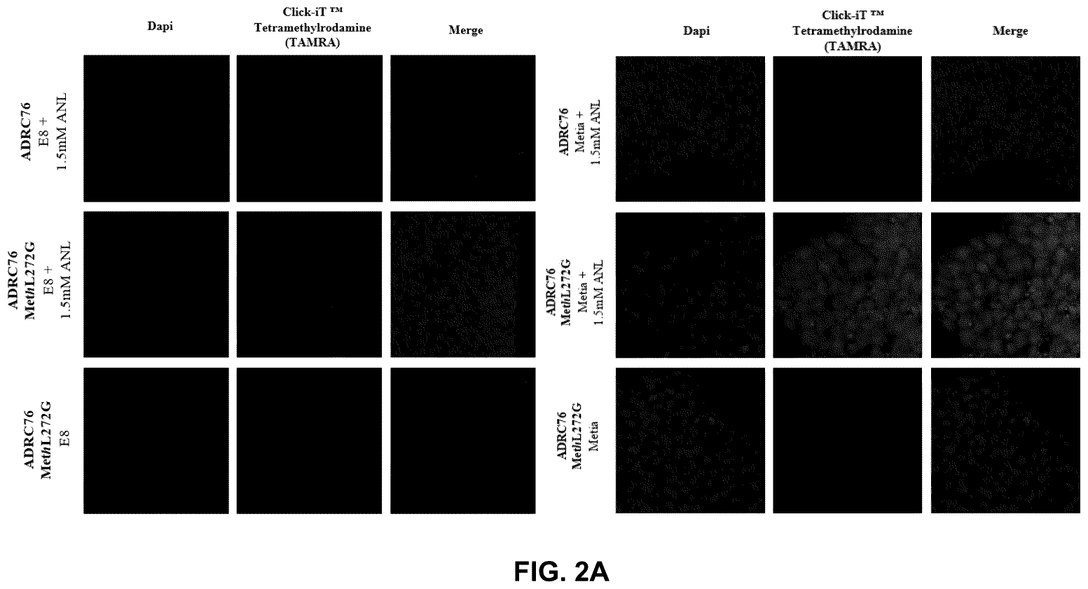

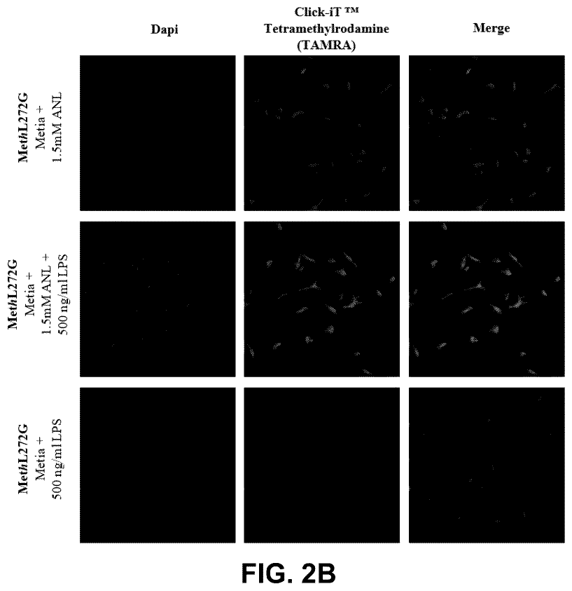

Validation in iPSCs: To validate ANL incorporation in iPSCs, the parental ADRC76 control and MethL272G iPSC lines were cultured on 8-well chambers with and without 1.5 mM ANL (Tocris 6585) for 14 hrs in TeSR™-E8™ media and DMEM/F12 Silac media without methionine (AthenaES 0433) supplemented with TeSR™-E8™ 25× Supplement. (Note: Methionine-absent media is described as Metia below.) Cells were fixed with 4% PFA, then treated with the Click-iT Protein Analysis TAMRA alkyne Detection Kit for confocal microscopy. Imaging demonstrates labeling of cellular protein only in mutated MethL272G iPSCs with greater labeling observed in cells grown in methionine-absent Media (FIG. 2A).

Validation of sensitivity to environmental changes (LPS addition to iMGL): To validate ANL incorporation in a differentiated lineage and the capacity to analyze the impact of environmental changes on protein synthesis utilizing the MethL272G cell line, microglia were derived from the MethL272G IPSO line and cultured in the presence and absence of lipopolysaccharide (LPS), a commonly used pro-inflammatory stimulus. Microglia were derived following the validated iPS-microglia 2.0 protocol outlined in McQuade et al (Mol Neurodegeneration, 2018) and cultured on 8-well chamber slides with 1.5 mM ANL in the presence and absence of 500 ng/ml LPS for 14 hrs in methionine-absent iMGL media. Differentiated iMGLs were fixed with 4% PFA, then treated with the Click-iT Protein Analysis TAMRA alkyne Detection Kit for confocal microscopy (FIG. 2B). Greater fluorescence was observed in the LPS treated MethL272G iMGLs indicative of the innate inflammatory response of microglia. Furthermore, imaging of the TAMRA-tagged protein of the treated iMGLs demonstrates the morphological change indicative of microglia activation as a result of LPS stimulation (FIG. 2B).

Labeling of Nascent Human RNAs:

Cloning and plasmids: A commonly used strategy for fitting bulky substrates into the active sites of enzymes is to create a corresponding ‘hole’ to the bulky ‘bump’ of large functional groups (‘bump-and-hole’). This strategy has been used successfully for many classes of enzymes and therefore this method was used to screen for TgUPRT mutants with different bulky modified uracil analogs. An exhaustive analysis of TgUPRT mutants is shown in Table 1 and corresponding complex uracil analogs that can provide a desired binary stringency for metabolic labeling of RNA.

The uprt gene, which encodes a protein annotated as uracil phosphoribosyltransferase from Toxoplasma gondii (European Nucleotide Archive code: AAB60213.1; UniProtKB Q26998) was amplified as Ndel-BamHI fragment from pKN342 containing wild-type (WT) of TgUPRT with 1×HA-tag at N-terminal (pET28a(+)-HA-TgUPRT-WT) to remove HA-tag existing in the template. The PCR product was then sub-cloned into the same expression vector pET28a(+) backbone, leading to the recombinant vector pKN-T7-TgUPRT-WT.

The different uprt mutants were generated by site-directed mutagenesis PCR from TgUPRT-WT. The resultant recombinant vectors are pKN-T7-TgUPRT-Mutt, pKN-T7-TgUPRT-Mut3, pKN-T7-TgUPRT-Mut4, pKN-T7-TgUPRT-Mut12 and pKN-T7-TgUPRT-Mut16 (See Table 1) provided the recombinant N-terminal 6×H is-tagged fusion proteins with a thrombin cleavage site between the tag and the enzyme to be used in vitro analysis. PCR amplicons of AsiSI and Mlul fragments from TgUPRT-WT and variants were sub-cloned into mammalian expression pCMV6 vector resulting recombinant vectors pKN-CMV-TgUPRT-WT and pKN-CMV-TgUPRT-Mutants (Mut) (Table 1).

Fragments of XbaI and XhoI mCherry were amplified from pRS35 template and sub-cloned into the pCDNA3.3 backbone, resulting in a pKN-CMV-mCherry recombinant plasmid.

Cell lines, bacterial and mammalian culture conditions: HEK293 cells (untransfected or TgUPRT-transiently transfected) were cultured in DMEM (Corning, Cat #: 10-017-CM) supplemented with 10% FBS, 1% (1 mg/mL) penicillin and streptomycin and grown at 37° C., 5% CO₂.

TgUPRT and TgUPRT variants were expressed in E. coli BL21(DE3) grown in LB medium at 37° C. with kanamycin 50 μg/mL. E. coli BL21(DE3) cell culture medium reagents were from Difco (St. Louis, United States). Trimethyl ammonium acetate buffer was purchased from Sigma-Aldrich (Madrid, Spain). All other reagents and organic solvents used in vitro studies were purchased from Scharlab (Barcelona, Spain) and Symta (Madrid, Spain). Nucleosides and nucleobases used in this work were provided by Carbosynth Ltd. (Compton, United Kingdom).

Transfection, labeling of cellular RNA and RNA isolation: After 48h seeding, HEK293 cells were transfected with 2.5 μg of TgUPRT WT or mutant plasmids (pKN-CMV-TgUPRT-WT or -Mutt to Mut17) per 5 cm plate using JetPrime Transfection reagent (Polyplus Transfection, France). At 40h post-transfection, cells were incubated with a final concentration of 200 μM at <1% DMSO from 400 mM stock of uracil analogs (5EU, 5VU, 5AU) or 200 mM SAMU stock for 5 h. For time course study, HEK cells were treated with 200 μM uracil analogs for 0 h, 0.5, 1, 3, 5, 12 and 24 hours. For titrating uracil analog concentration experiments, HEK cells were treated with 0 (DMSO), 50, 100, 200, 500 μM or 1 mM uracil analog for 5h. Treated HEK cells were subjected to total RNA extraction using 1 mL Trizol Reagent (Invitrogen) following the manufacturer instructions.

TABLE 1
TgUPRT plasmids and Description of Mutation.

| | | Description | |
|---|---|---|---|
| Plasmid ID | | Wild type Toxoplasma gondii Uracil | |
| pKN-CMV-TgUPRT-WT | | PhosphoRiboxylTransferase (TgUPRT-WT) | |
| Plasmid ID | Description | Plasmid ID | Description |

Under CMV promoter

| | | | |
|---|---|---|---|
| pKN-CMV-TgUPRT- | TgUPRT-M166A | pKN-CMV-TgUPRT- | TgUPRT-M166A-Y228G |
| Mut1 | | Mut11 | |
| pKN-CMV-TgUPRT- | TgUPRT-C167A | pKN-CMV-TgUPRT- | TgUPRT-A168G-Y228A |
| Mut2 | | Mut12 | |
| pKN-CMV-TgUPRT- | TgUPRT-A168G | pKN-CMV-TgUPRT- | TgUPRT-A168G-Y228G |
| Mut3 | | Mut13 | |
| pKN-CMV-TgUPRT- | TgUPRT-Y228A | pKN-CMV-TgUPRT- | TgUPRT-A168G-I229A |
| Mut4 | | Mut14 | |
| pKN-CMV-TgUPRT- | TgUPRT-Y228G | pKN-CMV-TgUPRT- | TgUPRT-A168G-I229G |
| Mut5 | | Mut15 | |
| pKN-CMV-TgUPRT- | TgUPRT-I229A | pKN-CMV-TgUPRT- | TgUPRT-M166A-A168G-Y |
| Mut6 | | Mut16 | 228A |
| pKN-CMV-TgUPRT- | TgUPRT-I229G | pKN-CMV-TgUPRT- | TgUPRT-M166A-A168G-Y |
| Mut7 | | Mut17 | 228G |
| pKN-CMV-TgUPRT- | TgUPRT-D235A | pKN-CMV-EGFP-Tg | EGFP-TgUPRT-WT |
| Mut8 | | UPRT-WT | |
| pKN-CMV-TgUPRT- | TgUPRT-M166A-A168G | pKN-CMV-EGFP-Tg | EGFP-TgUPRT-M166A-A |
| Mut9 | | UPRT-Mut16 | 168G-Y228A |
| pKN-CMV-TgUPRT- | TgUPRT-M166A-Y228A | pKN-CMV-EGFP-Tg | EGFP-TgUPRT-M166A-A |
| Mut10 | | UPRT-Mut17 | 168G-Y228G |
| pKN-CMV-mCherry | mCherry | pKN-CMV-EGFP | pCDNA3.3-EGFP |
| | | | (Addgene, Cat#) |

Under T7 promoter

| | | | |
|---|---|---|---|
| pKN-T7-TgUPRT-WT | TgUPRT-WT | pKN-T7-TgUPRT- | TgUPRT-Y228A |
| | | Mut4 | |
| pKN-T7-TgUPRT- | TgUPRT-M166A | pKN-T7-TgUPRT- | TgUPRT-A168G-Y228A |
| Mut1 | | Mut12 | |
| pKN-T7-TgUPRT- | TgUPRT-A168G | pKN-T7-TgUPRT- | TgUPRT-M166A-A168G-Y |
| Mut3 | | Mut16 | 228A |

Co-culture of HEK cells containing GFP or mCherry expression plasmids: HEK293 (5×105) cells seeded in 6-well plate. Thirty-six hour post seeding, HEK cells were transfected with 1 μg of single plasmid containing EGFP, mCherry, EGFP-TgUPRT-WT, or EGFP-TgUPRT-Mut. Twenty-four hour post transfection, transiently transfected HEK cells were trypsinized and seeded into a 5 cm plate. Equal amounts of cells (approximately 2×106 in total) where 1×106 cells containing GFP or GFP-TgUPRT plasmids and 1×106 cells containing mCherry construct) were co-cultured. Three co-culture conditions are Condition (A): mCherry and EGFP, Condition (B): mCherry and EGFP-WT-TgUPRT, and Condition (C): mCherry and EGFP-3×MT-TgUPRT (pKN-CMV-EGFP-TgUPRT-Mut16 or -Mut17). Sixteen hours post seeding, the co-cultures were treated with 5EU and 5VU for 5 hours at 200 μM and 500 μM final concentration, respectively. Cell imaging was performed to assess the overall distribution of green and red cells in each co-culture. Once imaging was done, cells were harvested and subjected to RNA isolation. Total RNA isolated from the co-culture was subjected to biotinylation by two click methods: (1) CuAAC to assess 5EU incorporation in RNA and (2) IEDDA to assess 5VU incorporation in RNA. Subsequently, biotinylated RNA was subjected to dot blot analysis.

Biotinylation via CuAAC and IEDDA: Cu-mediated Azide-Alkyne cycloaddition (CuAAC) click reactions were performed in 50 μL reaction at 22° C. on a shaker for 30 min with click reaction cocktail containing 15 μg of total RNA, 1 mM biotin alkyne or biotin azide, fresh 4.6 mM THPTA to a final concentration of 1 mM, and fresh 10.6 mM sodium ascorbate (NaAsc) at final concentration of 1.77 mM, and 12 mM CuSO4 at final concentration of 200 μM in biotinylation reaction buffer (10 mM TrisHCI, 1 mM EDTA, pH7.4). The biotinylated RNA was purified using RNA clean & Concentrator-5 kit (Zymo Research, Cat #R1013) according to the manufacturer instructions and eluted in 21 μL of nuclease free water.

Inverse electron demand [4+2] Diels-Alder cycloaddition (IEDDA) click reactions were performed in 50 μL at 37° C. or 22° C. (RT) for 3h on mixer at 500 rpm containing 10-15 μg of total RNA, 1 mM final concentration of biotin-benzyl tetrazine (Sigma Aldrich, Cat #793329-5MG) in nuclease free water. Clicked total RNA was purified using RNA clean & Concentrator-5 kit (Zymo Research, Cat #R1013) according to the manufacturer instructions, and RNA was eluted in 21 μL of nuclease free water.

HRP-streptavidin dot blot analysis: All gel reagents were from Bio-Rad. For dot blot analysis, 2 μg of clicked total RNA was applied onto Hybond-N+ membrane (GE Healthcare) as individual dots and UV-crosslinked (254 nm) to a membrane (Stratalinker UV crosslinker). Membranes were blocked in blocking buffer (0.12 M NaCl, 0.016 M Na2HPO4, 0.008 M NaH2PO4, 0.17 M SDS) and followed by incubation with high sensitivity streptavidin-HRP (Fisher Scientific, Cat #: P121130) at 1:5000 dilution in blocking buffer for 5 minutes. The membrane was washed twice in a Wash A buffer (1:10 dilution of blocking buffer) and twice in Wash B buffer (Tris-saline buffer). It was then incubated for 1-5 min. in ECL Chemiluminescent Substrate (Fisher Scientific, Cat #: P132106) and imaged on a ChemiDoc MP imaging system (Bio-Rad).

RNA fluorescence imaging via IEDDA: The cell culture dishes and glass coverslips were coated with poly-D-lysine (10 μg/ml) for 24 h at 37° C. and washed three times with autoclaved water to remove the excessive amount of poly-D lysine. HEK293 cells were seeded on glass coverslips at 2.5×105 in a 6-well plate. Twenty-four hours post-seeding, cells were transiently transfected with 1 μg WT- or MT-TgUPRT using jetPRIME transfection reagent according to the manufacturers manual (Polyplus Transfection, France). Twenty-four hours post-transfection, cells were treated with DMSO or 1 mM 5VU for 24 hours. After labeling, cells were washed 3× with DPBS, fixed and permeabilized for 30 min at room temperature with 3.7% paraformaldehyde and 0.15% Triton-X100. Cells were then washed three times (7 min/each) on orbital shaker with DPBS, blocked with Streptavidin (2 mg/mL in DPBS) for 30 minutes and followed with BSA (1 mg/ml in DPBS, 0.45% NaCl and 0.025% NaN3) for 30 min at room temperature, washed 2× with DPBS. Blocked cells were incubated with 200-500 μL of 1 mM biotin-tetrazine solution in water for 3 hours at 37° C. Cells were washed 4× times for 5 min/each on an orbital shaker: twice with 1 mL of DPBS+0.1% Triton-X100 and twice with 1 mL of DPBS on shaker for 5 min/each. Upon removal of DPBS, cells were incubated with 200-300 μL of Streptavidin-Alexa-488 (2 mM fluorophore stock at 1:1000 dilution in DPBS) for 30 minutes at room temperature in a light shield container. Cells were wash 3× with DPBS+0.1% Triton for 5 min/ea & 1× DPBS (1 ml/ea) on shaker then stained with Hoechst 333242 (at 1:2000 dilution in DPBS) for 10 min., washed 3× with DPBS for 5 min/each and mounted using VectaShield antifade (Vector Labs). Slides were imaged via fluorescence confocal microscopy using a 63× oil immersion objective on a Leica 700 Carl Zeiss microscope.

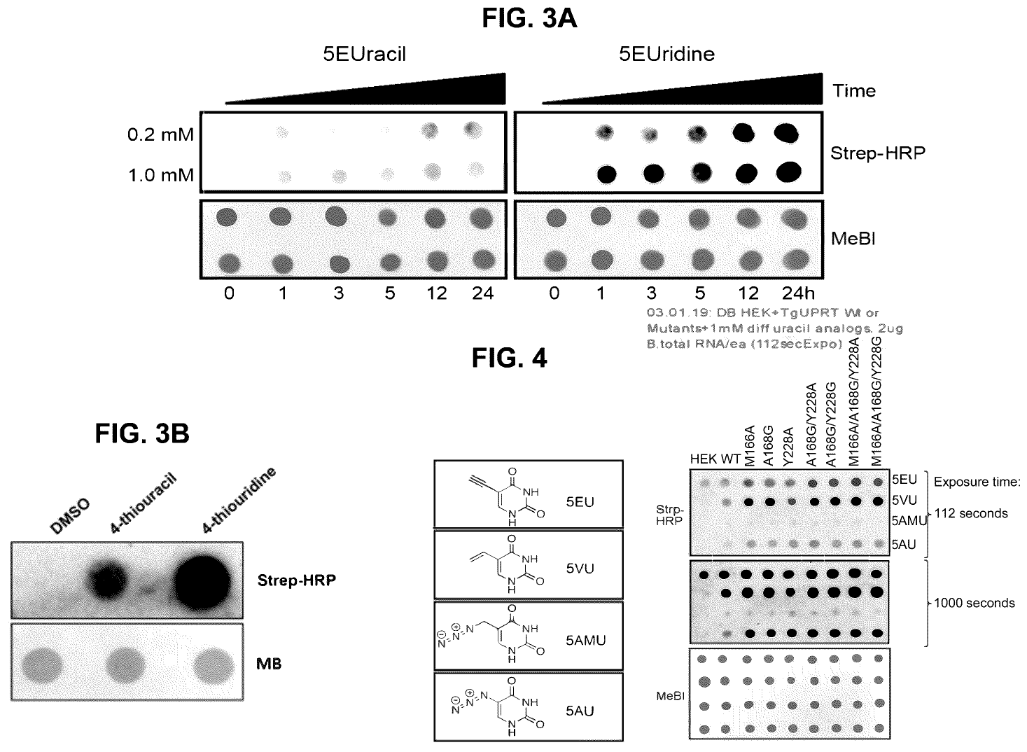

Protein isolation and Immunoblot analysis: For a time course study (FIG. 3A), HEK cells grown in a 6-well plate and transfected in duplicates with each 1.0 μg per WT- or MT-TgUPRT construct. At twenty-four hours post transfection (24h PTF), at 36h, 43h, 45h, 47h and 47.5h PTF, 200 μM 5VU was added to the two wells for corresponding time points 24, 12, 5, 3, 1 and 0.5 hours, respectively. For 0 h, equal volume of DMSO carrier was added for 24h treatment. At 48h PTF, cells from both wells of the same condition of treatment were washed, scraped off and transferred to a 1.5 mL microfuge tube. The mixture of cells was divided in half and pelleted: (1) for RNA isolation as described in the RNA isolation section, and (2) for total protein extraction (whole cell extract-WCE).

To each cell pellet, 200 μL RIPA lysis buffer, supplemented with 4 μL of proteinase inhibitor cocktail Set VII (EMB, Cat #: 539138-1 ML), 2 μL Turbo DNase (Life Tech., Cat #AM2239), 2 μL RNaseA (Thermo Fisher, Cat #FEREN0531) per milliliter of lysis buffer, was added. Cells were resuspended and incubated on ice for 0.5h then sonicated for 11 seconds (5 seconds ON, 1 second OFF) at 4° C. WCE was cleared by centrifugation at 15K rpm for 30 min at 4° C. The cleared lysate was subjected to BCA assay (BioSciences, Cat #786-570) to determine protein concentration according to Manufacturer instruction at 37° C. for 30 min and quantified using NanoDrop BCA program. SDS-PAGE analysis was performed: 2 μg of total protein was resolved in 4-20% gradient 10-well MiniProtean gel (Bio-Rad, Cat #4561094).

Proteins were transferred to nitrocellulose membrane using Trans-Blot Turbo Transfer unit (Bio-Rad, Cat #1704150). Membranes were blocked with 1×PBST (1×PBS+0.1% Tween-20)+5% non-fat milk for 1 h at room temperature and probed overnight at 4° C. with fresh blocking buffer+mouse anti-6×His (GeneScript, Cat #: A00186-100) at 1 μg/mL. Blot was washed 3×10 min/each with 1×PBST at RT and incubated in 1×PBST+antibody of anti-mouse conjugated Horseradish peroxidase (1:20000 dilution) (Cell Signaling, Cat #7076S) for 1 h at room temperature. Finally, blot was washed 3×10 min/each with 1×PBST at RT and imaged using ECL Chemiluminescent Substrate (Fisher Scientific, Cat #P132106). Protein blots were subsequently stained in Pierce PageBlue Protein staining solution (Fisher Scientific, Cat #: P124620) and imaged on a ChemiDoc MP imaging system (Bio-Rad).

Expression and Purification recombinant enzymes for in vitro analysis: Protein overexpression was induced by adding 0.5 mM isopropyl β-D-1-thiogalactopyranoside and the cells were further grown for 3 hours. These were harvested via centrifugation at 3500×g. The resulting pellet was resuspended in 10 mM sodium phosphate buffer pH 7. Crude extracts were prepared by French press lysis of cell suspensions. The lysate was centrifuged at 17500×g for 30 min and the supernatant was filtered through a 0.22 μm filter (Millipore). The cleared lysate was loaded onto a 5-mL HisTrap FF column (GE Healthcare) pre-equilibrated in a binding buffer (20 mM Tris-HCl buffer, pH 8.0, with 100 mM NaCl and 10 mM imidazole) and the column was washed. Bound proteins were eluted using a linear gradient of imidazole (from 10 to 500 mM). Fractions containing recombinant enzyme were identified by SDS-PAGE, pooled, concentrated and loaded onto a HiLoad 16/60 Superdex 200 prep grade column (GE Healthcare) pre-equilibrated in 20 mM Tris-HCl buffer, pH 7.0. Fractions with the protein of interest identified by SDS-PAGE were pooled and the protein was dialyzed against 10 mM sodium phosphate, pH 7.0, and concentrated and stored at 4° C. until its use. Electrophoresis was carried out on a 15% polyacrylamide slab gel with 25 mM Tris-HCl buffer, pH 8.6, 0.1% SDS. Protein concentration was determined spectrophotometrically by UV absorption at 280 nm using corresponding molar extinction values (ε₂₈₀=17,795-19285 M−1 cm−1.)

Enzyme activity assay: The standard activity assay was performed by incubating 10 μL of free extract or 0.5 μg of pure His-tag enzyme with 1 mM Phosphoribosyl pyrophosphate (PRPP), 1 mM uracil, 5 mM MgCl2 in 50 mM PBS buffer pH 7.4 in a final volume of 80 μL. The reaction mixture was incubated at 37° C. for 2-5 min (300 rpm). Enzyme was inactivated by adding 80 μL of cold methanol in ice-bath and heating for 5 min at 100° C. After centrifugation at 9000×g for 5 min, samples were half-diluted with water and the NMP production was analysed by HPLC to quantitatively measure the reaction products as described below in the analytical methods. All determinations were carried out in triplicate assuming Michaelis-Menten conditions, and the maximum error was below 5%. In such conditions, one international activity unit (IU) was defined as the amount of enzyme producing 1 μmol/min of UMP under the assay conditions.

Substrate specificity studies Enzymatic synthesis of the different pyrimidine nucleoside monophosphate (NMP) derivatives was carried out by incubating 0.5 μg of pure His-tag enzyme with 1 mM PRPP, 1 mM pyrimidine base and 5 mM MgCl2 in 50 mM PBS buffer pH 7.4 (1.6-3.2% v/v DSMO). The experiments were performed according to the standard assay. Subsequently, the reaction mixture was processed as described above and the NMP production was analyzed by HPLC.

Analytical methods: The production of NMPs was quantitatively measured with an ACE EXCEL 5 μm CN-ES 250×4.6 mm equilibrated with 100% trimethyl ammonium acetate at a flow rate of 0.8 mL/min. Retention times for the reference bases and NMPs (hereafter abbreviated according to the recommendations of the IUPAC-IUB Commission on Biochemical Nomenclature) were as follows: uracil (Ura), 4.7 min; 5-azido uracil (5-AUra). 13.3 min; 5-azido methyl uracil (5AMUra), 6.6 min; 5-ethynyl uracil (5-EtUra). 7.5 min; 5-vinyl uracil (5-VUra), 16.0; uridine-5′-monophosphate (UMP), 3.6 min; 5-azido uridine-5′-monophosphate (5-AUMP), 5.1; 5-ethynyl uridine-5′-monophosphate (5-EtUMP). 4.0 min; 5-vinyl uridne-5′-monophosphate (5-VUMP), 4.2 min. Results were normalized based on the nucleobase mass balance.

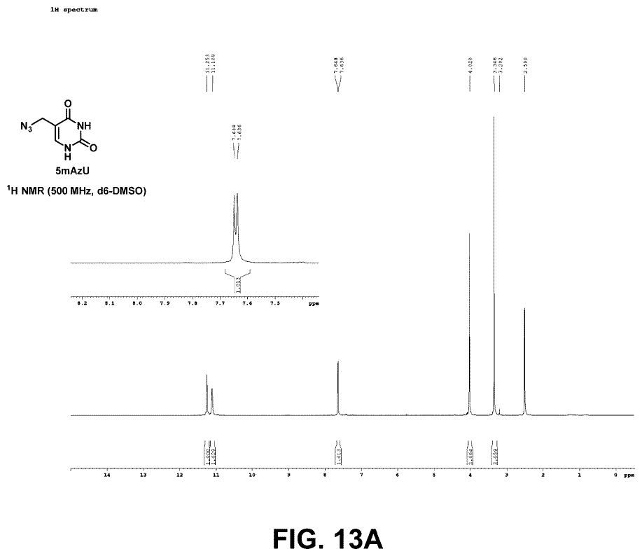

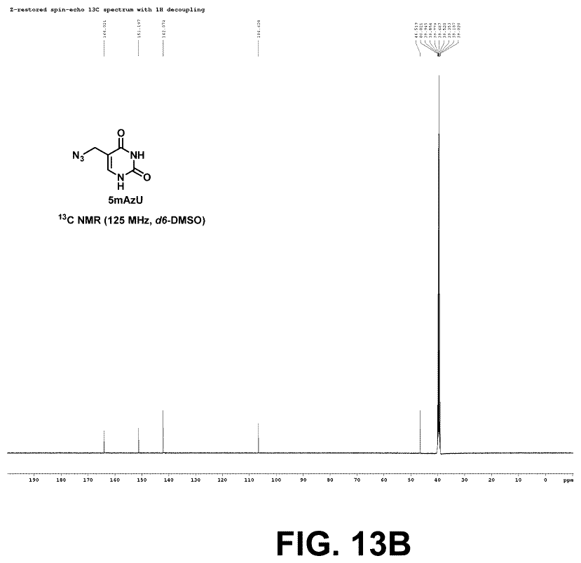

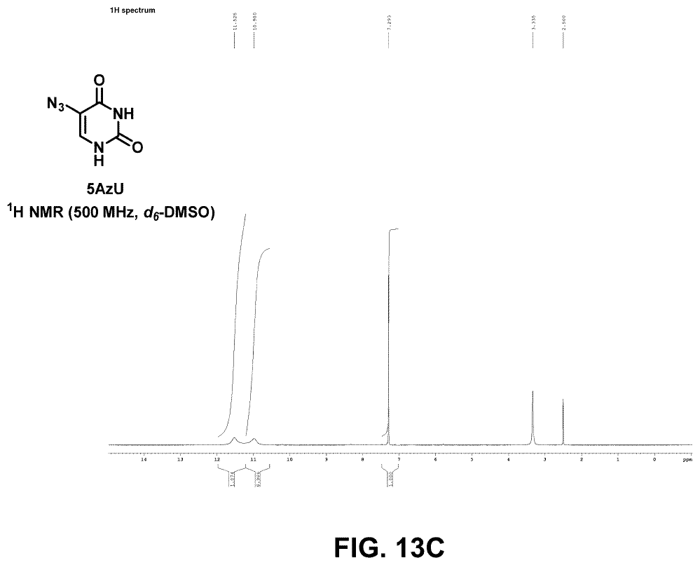

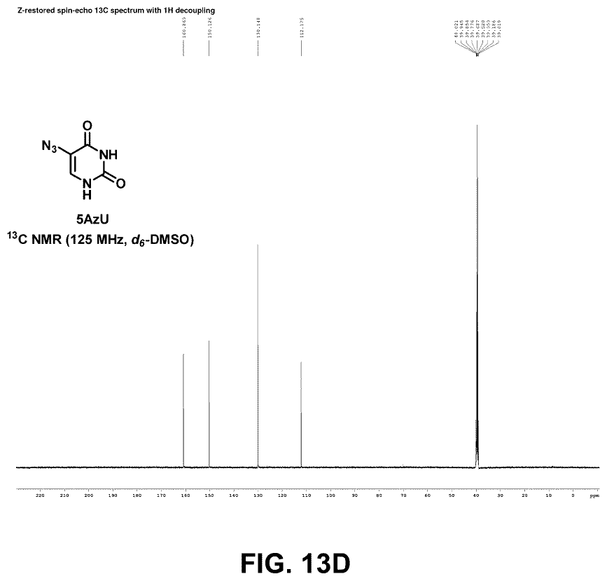

Synthesis and spectra of RNA Analogs (FIGS. 13A, 13B. 13C, and 13D)

5-ethynylpyrimidine-2,4(1H,3H)-dione (5EU)

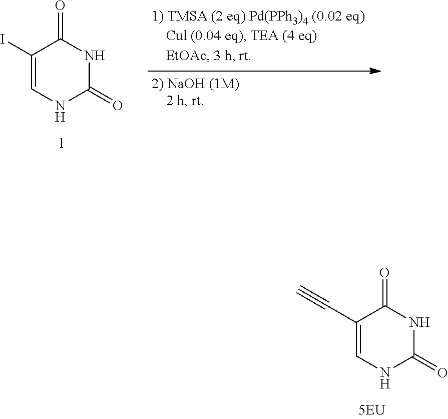

The product was prepared accordingly, 5-lodouracil (2000 mg, 8.4 mmol, 1 eq). TMS-acetylene (2.4 mL, 16.8 mmol, 2 eq), Et₃N (4.7 mL, 33.6 mmol, 4 eq). Pd(PPh₃)₄ (196 mg, 0.17 mmol, 0.02 eq), and Cut (65 mg, 0.34 mmol, 0.04 eq) were dissolved in 25 mL of degassed EtOAc. The suspension was stirred at room temperature for 3 hrs under Ar. The suspension was then filtered and washed with EtOAc. The extract was collected and dissolved in 10 mL of 1 M NaOH and stirred at room temperature for 2 hrs. The solution was then diluted with 10 mL of H₂O and concentrated in vacuo. The residue was then redissolved in 10 mL of H₂O and AcOH was added until a pH of 5 was reached. The suspension was then set on ice for 30 mins and filtered. The extract was washed with H₂O, acetone, and Et₂O. The extract was then dried in vacuo to give 5-ethynylpyrimidine-2,4(1H,3H)-dione (823 mg, 72%) as an off white solid. HRMS Calcd for C₈H₄N₂O₂[M⁺] 136.11, found 135.02 [M−H⁻]; ¹H NMR (400 MHz, DMSO) δ 11.29 (s. 2H), 7.78 (s, 1H), 3.99 (s, 1H). ¹³C NMR (126 MHz, DMSO) δ 163.22, 150.96, 147.02, 96.72, 83.71, 77.08.

5-Vinylpyrimidine-2,4(1H,3H)-dione (5VU)

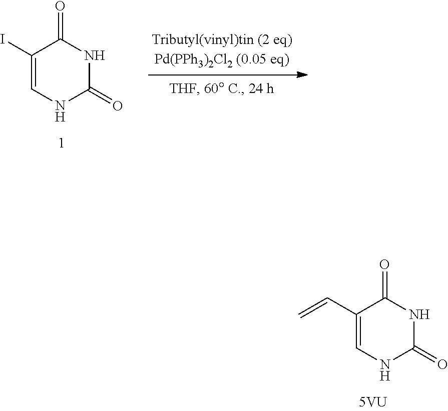

To a solution of 5-iodopyrimidine-2.4(1H,3H)-dione (1) (10 g, 4.20 mmol, 1 eq) and Pd(PPh3)2Cl2 (147.4 mg, 0.210 mmol, 0.05 eq) in dry THF (15 mL) was added tributyl(vinyl) stannane (2.45 mL, 8.40 mmol, 2 eq) dropwise at room temperature under argon. The resulting reaction mixture was warmed up to 60° C. and stirred at 60° C. for 24 h. The reaction mixture was then cooled down to room temperature and filtered through a pad of celite. washed with MeOH, concentrated to afford a yellowish slurry, which was recrystallized with Hexane/MeOH to afford 2 as a pale yellow solid (205.0 mg, 35% yield). 1H NMR (600 MHz, d6-DMSO) δ 5.06 (d, J=11.4 Hz, 13 H), 5.95 (d, J=18 Hz, 1H), 6.35 (dd, J=18, 11.4 Hz, 1H), 7.58 (s, 1H), 11.04 (s, 1H), 11.13 (s, 1H); 13C NMR (150 MHz, d6-DMSO) δ 109.6, 113.4, 129.0, 139.9, 150.6, 163.1; HRMS (ESI) calculated for C6H6N2O2Na [(M+Na)+] 161.0327, found 161.0333.

5-Azidopyrimidine-2,4(1H,3H)-dione (5AzU). 5AzU was synthesized according to a reported procedure.

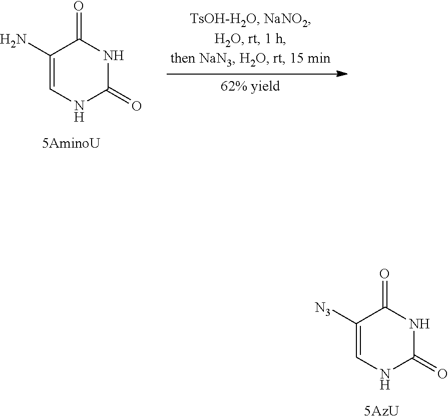 [SMILES: Nc1c[nH]c(=O)[nH]c1=O ; [N-]=[N+]=Nc1c[nH]c(=O)[nH]c1=O]

To a solution of 5-aminopyrimidine-2,4(1H,3H)-dione (5AminoU) (2.25 g, 17.7 mmol) in water (150 mL) at room temperature was added p-toluenesulfonic acid monohydrate (30.3 g, 160 mmol) and sodium nitric (11.0 g, 160 mmol) slowly and the reaction mixture was stirred vigorously at room temperature for 1 h. Sodium azide (1.84 g, 28.4 mmol) was added slowly to the reaction mixture and the reaction was stirred for an additional 15 min until the bubbling was gone. The resulting reaction mixture was cooled down on an ice bath to precipitate out the product 5AzU, which was obtained by filtration. After washing by water twice and diethyl ether twice, 5AzU was obtained as light beige solid (1.67 g, 62% yield) 1H NMR (500 MHz, d6-DMSO) δ 7.29 (s, 1H), 11.0 (brs, 1H), 11.5 (brs, 1H) ppm; 13C NMR (125 MHz, d6-DMSO) δ 112.2, 130.1, 150.1, 160.9 ppm. The 1H and 13C NMR spectra were identical to those reported.

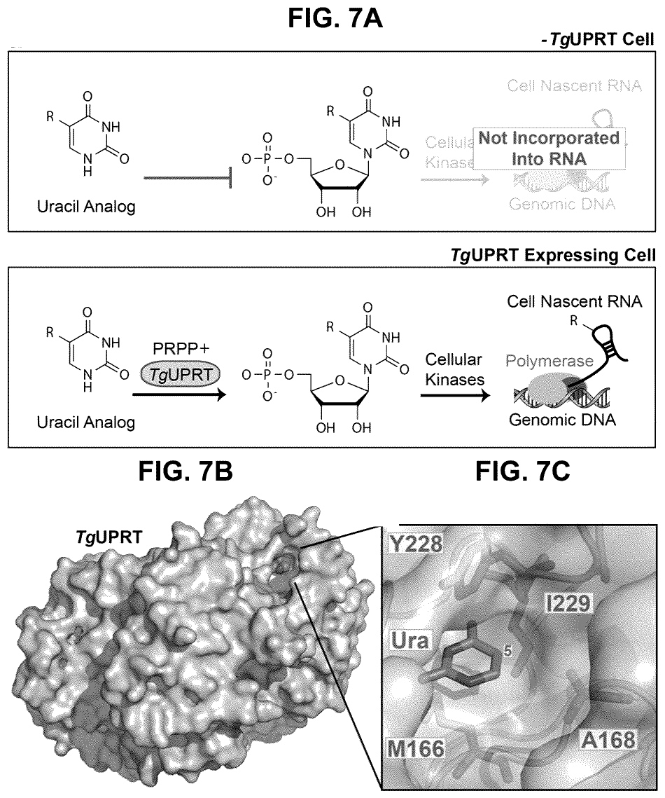

The active site of TgUPRT was explored and several active site residues identified that could be amenable to mutation (FIGS. 7B and 7C). As shown in FIGS. 3A and 3B, the most widely adopted uracil analog, 4-thiouracil, has the ability to be incorporated into cellular RNA, in a TgUPRT-dependent manner. 5-ethynyluracil (FIG. 3A) was capable of such transformations (and in (−)TgUPRT cells and also has a low level of background incorporation in (−)TgUPRT cells (FIG. 3B). However, uracil analogs with bulkier functional groups, such as vinyl and methylazido were not, presumably due to steric clashes with the TgUPRT active site. Inspection of the TgUPRT crystal structure in complex with uracil supports this notion as the uracil nucleobase is tightly surrounded by many amino acid residues which likely clash with bulkier functional groups at the 5-position on uracil (FIG. 7B).

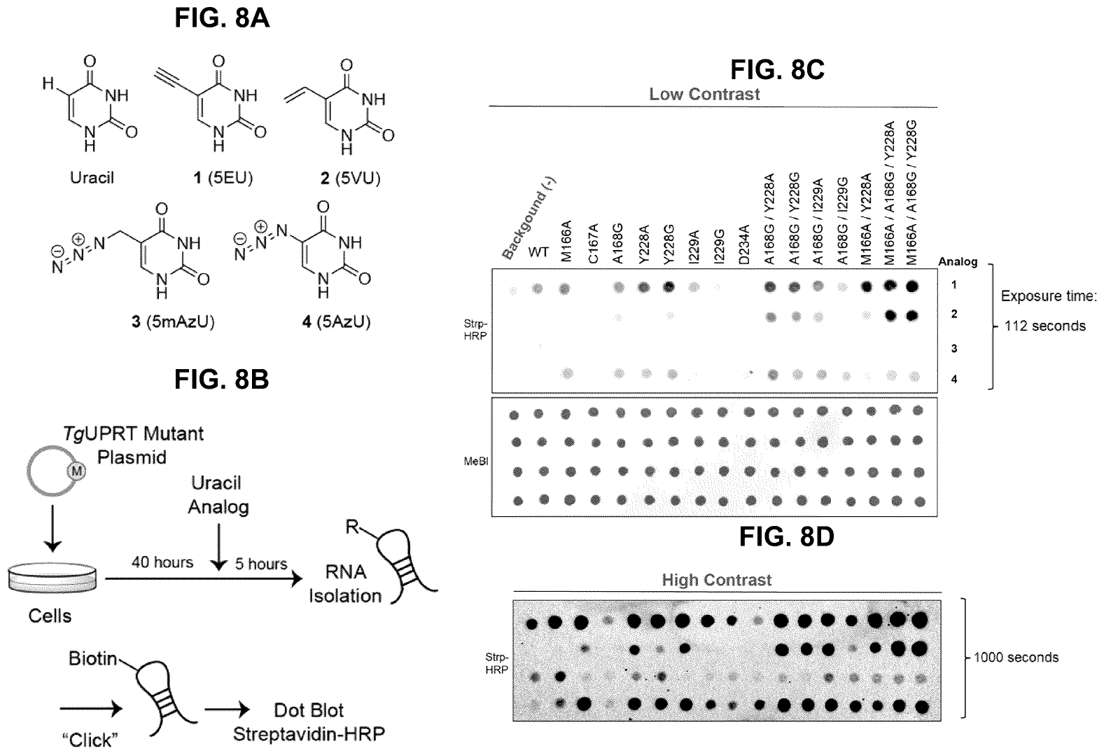

To complement the mutants, four variants of uracil were designed and synthesized with differing functional group complexity at position 5 (FIG. 8A). Single, double and triple mutations were cloned and transiently transfected into HEK293T cells. 40 hours post transfection, each uracil analog was added at 200 μM final concentration for 5 hours. RNA was subsequently isolated and appended with biotin using either copper-catalyzed azide-alkyne cycloaddition (CuAAC), with biotin conjugated alkyne or azide (for azido- and alkynyl-uracil analogs). For analog 2 (5VU), an inverse electron-demand Diels-Alder was utilized with a biotin-conjugated tetrazene (IEDDA). Following bioorthogonal ligation, biotinylation of RNA was assayed using streptavidin-HRP dot blot (FIG. 8B). As shown in FIG. 8C, background incorporation of 1 (5EU) was observed at this moderate concentration. Notably, at higher concentrations that mimic in vivo applications (1 mM), evidence of incorporation of 2 was not observed (FIG. 4), but very robust incorporation of 1. In addition, very slight evidence of incorporation of 3 (5mAzU), with higher incorporation of 4 (5AzU) was observed.

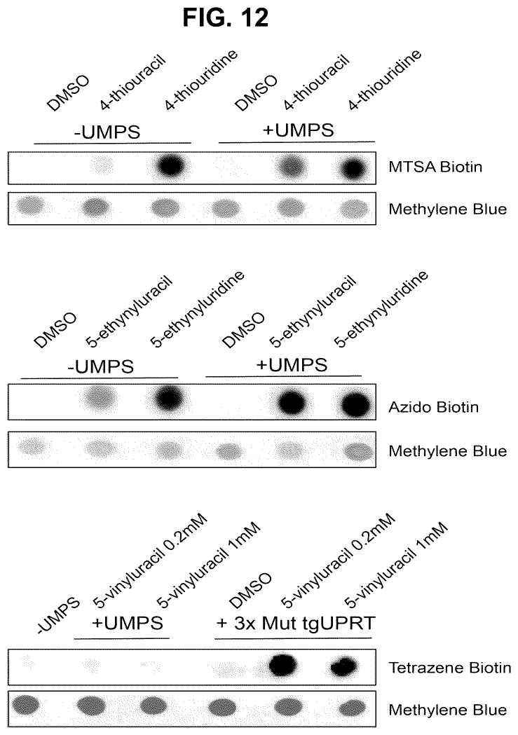

From the comparison of mutant-dependent uracil analog incorporation in RNA of cells containing mutant TgUPRT to (−)TgUPRT (un-transfected) cells and the wild-type (WT) TgUPRT, several mutants seemed to be compatible with most C-5 modified uracil analogs and that the triple mutants (3×MT), M166A/A168G/Y228A and M166A/A168G/Y228G enabled robust incorporation of 5-vinyluracil (5VU). Trypan blue measurements, for cell viability, also demonstrated that this pair (the triple mutant and 5VU) exhibits non-significant differences to untreated cells (FIG. 11), consistent with recent evaluations with 5-vinyluridine analogs. In previous work, it was demonstrated that background labeling of uracil and uracil-like compounds was due to the expression of uracil monophosphate synthetase (UMPS). It was also demonstrated that overexpression of UMPS results in a higher amount of 1 and 4-thiouracil incorporation into cellular RNA. However, UMPS overexpression still did not result in 2 incorporation into RNA (FIG. 12).

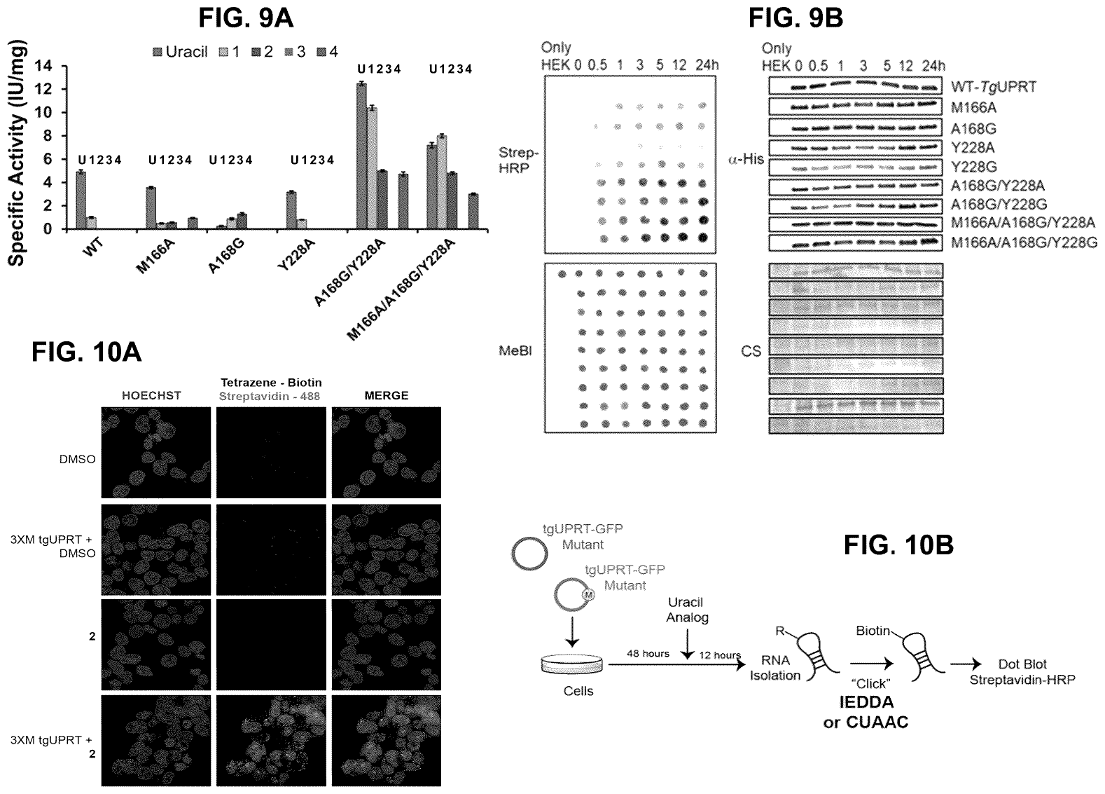

These results further suggest that 2 is not a viable substrate for endogenous enzyme pathways for eventual incorporation into RNA. Following these exciting observations, a more quantitative approach to examined for differences between enzyme mutants and their enzyme kinetics with the analogs. Five recombinant mutants were expressed and purified as well as Wild Type (WT) TgUPRT (Table 2). Each purified enzyme was incubated with the different substrates and phosphoribosyltransferase activity was measured (FIG. 9A). As shown, reactivity of uracil for all five mutants was observed, but at different level of specific activity. Consistent with in-cell screening, compound 2 has undetectable activity in WT but increased dramatically with mutants, whereas compound 1 was reactive with WT enzymes, while also exhibiting increased reactivity with the mutants (10 IU/mg). Overall, these results support in-cell screening efforts and nicely demonstrate increased reactivity with mutant-analog pairs (2 and mutants TgUPRT) that have much higher specificity and undetectable background activity with WT enzymes.

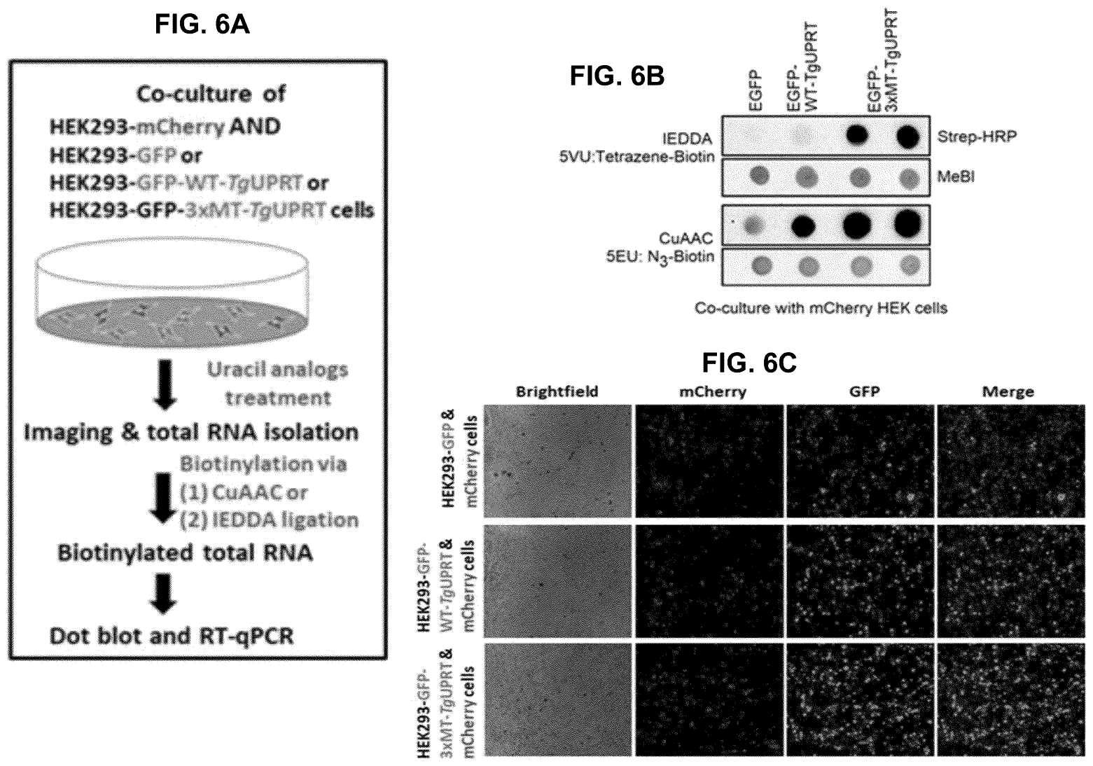

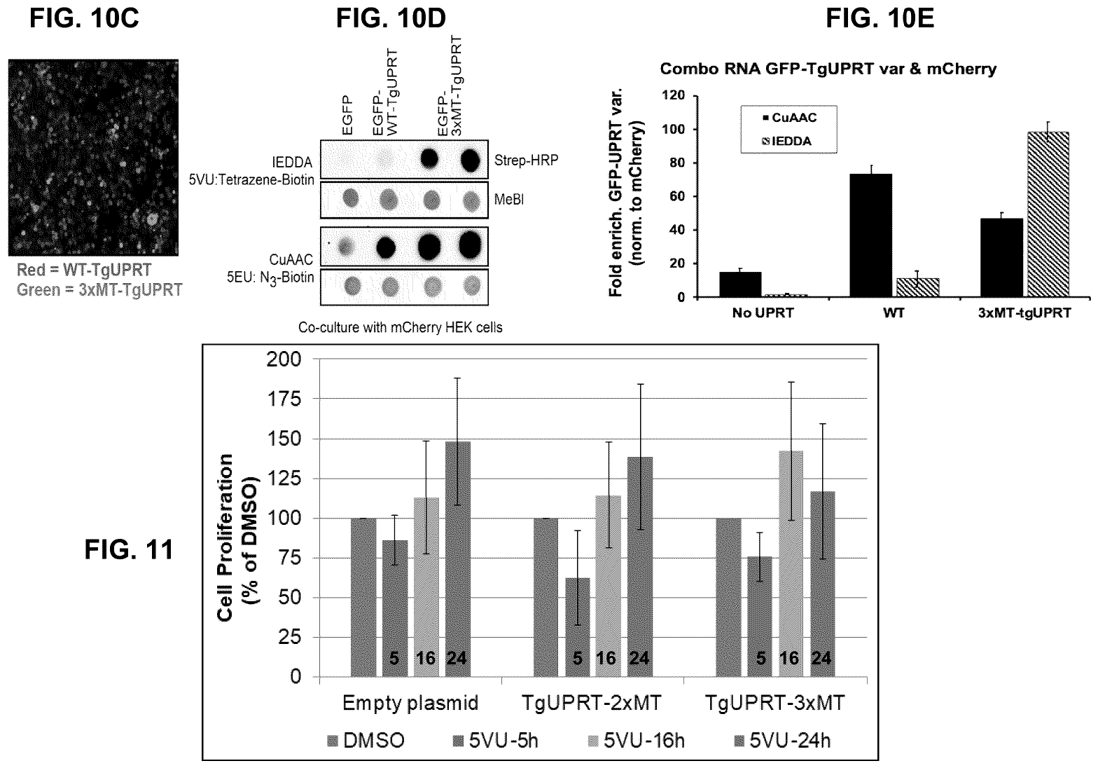

Using dot blot analysis to evaluate the level of 5-vinyluracil incorporation into RNA, the analog incorporation was also observed in as little as 0.5 hr, which is faster and more robust than previously reported. Analog incorporation was much more robust over time with mutant TgUPRT enzymes, in comparison to the wild-type, which over long periods of incubation with 2 did not result in any observed incorporation (FIG. 9B). Overall, these results further support the specificity of the designed mutants and also demonstrate the increased metabolic incorporation and efficiency of the triple mutant TgUPRT/2 pair. Imaging experiments were performed to further demonstrate stringency at higher concentrations that mimic those used in vivo. Briefly, 2 was incubated with cells for 24 hours at 1 mM concentration. Cells were fixed and permeabilized and incubated with tetrazene biotin (for inverse electron-demand Diels-Alder appending of biotin). Following, fluorophore-conjugated streptavidin was incubated with cells and imaged. As shown in FIG. 10A, fluorescent signal was observed only in the presence of both 2 and 3×MT-TgUPRT transfected cells but not in cells in the experiments with either 2 or mutant TgUPRT alone. Lastly, to stringently test specificity of the bioorthogonal pair of 3×MT-TgUPRT and 2, two population of HEK293T cells: (1) singly transfected with mCherry containing plasmid and (2) singly transfected with GFP-3×MT-TgUPRT or GFP-WT-TgUPRT vector, were co-cultured and treated with both 200 μM 5EU and 500 μM 5VU or with DMSO for 5 hours (FIG. 10B; FIG. 6A). The co-culture was imaged (FIG. 10C). The cells were subjected to RNA isolation followed by biotinylation with biotin conjugated tetrazine or alkyne (FIG. 10D). Assessment of 5EU vs. 5VU incorporation into RNA using biotinylated total RNA from the co-culture with streptavidin-HRP in dot blot analysis demonstrates detectable signal of 5EU in the absence of TgUPRT. In great contrast, GFP-3×MT-TgUPRT mediated 5VU incorporation into RNA, substantially. The enrichment of GFP transcript in co-culture experiments was compared; compared the fold-enrichment to mCherry. Background enrichment was observed in the -UPRT cells, further demonstrating 5EU is incorporated into RNA in background cells. When comparing triple mutant cells to the no UPRT cells, nearly 100-fold enrichment of GFP transcript was observed only when 3×MT-TgUPRT enzyme cells were incubated with 2. These results clearly demonstrate the other uracil analogs have background RNA incorporation which can result in enrichment of transcripts in off-target cells. In contrast, the new 3×MT-TgUPRT/2 pair, described herein, is highly stringent and can be used to enrich RNAs specifically from target cells.

TABLE 2
Specific activity analysis of TgUPRT variants with different uracil analog substrates in vitro:

| | Specific Activity (IU/mg) | | | | | |
|---|---|---|---|---|---|---|
| | | TgUPRT | TgUPRT | TgUPRT | TgUPRT | TgUPRT |
| | TgUPRT | A186G | M166A | Y228A | A168G/Y228A | M166A/A168G/Y228A |
| Uracil^{a} | 4.92 ∓ 0.17 | 0.26 ∓ 0.05 | 3.57 ∓ 0.09 | 3.18 ∓ 0.11 | 12.49 ∓ 0.18 | 7.20 ∓ 0.23 |
| 5-ethynly uracil | 1.00 ∓ 0.08 | 0.88 ∓ 0.09 | 0.48 ∓ 0.05 | 0.80 ∓ 0.03 | 10.40 ∓ 0.22 | 8.00 ∓ 0.17 |
| (5-EtUra)^{b} | | | | | | |
| 5-vinyl uracil | n.d. | 1.30 ∓ 0.12 | 0.56 ∓ 0.06 | n.d. | 5.0 ∓ 0.09 | 4.80 ∓ 0.12 |
| (5-VUra)^{c} | | | | | | |
| 5-azido uracil | n.d. | n.d. | 0.95 ∓ 0.05 | n.d. | 4.72 ∓ 0.19 | 3.01 ∓ 0.09 |
| (5-AU)^{b} | | | | | | |
| 5-azido methyl uracil | n.d. | n.d. | n.d. | n.d. | n.d. | n.d. |
| (5AMU)^{b} | | | | | | |
| 5-bromovinyl uracil | 0.10 ∓ 0.01 | 0.12 ∓ 0.03 | 0.10 ∓ 0.02 | 0.10 ∓ 0.01 | 2.80 ∓ 0.25 | 2.34 ∓ 0.15 |
| (5-BrVUra)^{a} | | | | | | |

Enzymatic synthesis of uridine-5′-monophosphate analogues catalyzed by TgUPRT:
^{a}Reaction conditions: 0.5 μg of enzyme in 80 μL at 37° C., 2-5 min. [Substrates] = 1 mM, in PBS pH 7.4
^{b}Reaction conditions: 0.5 μg of enzyme in 80 μL at 37° C., 2-5 min. [Substrates] = 1 mM, in PBS pH 7.4 (1.6% DMSO)
^{c}Reaction conditions: 0.5 μg of enzyme in 80 μL at 37° C., 2-5 min. [Substrates] = 1 mM, in PBS pH 7.4 (3.2% DMSO)
n.d. = not detected.

Overall, the present invention features a novel nucleobase-enzyme pair for highly-stringent and cell-specific metabolic labeling of RNA. The present invention is appreciated to have reduced background issues due to endogenous enzymatic and metabolic activities. These findings can be extended to living animal settings.

Furthermore, the present invention has developed a novel mutant UPRT enzyme—modified analog (5-vinyluracil) which has a higher specificity index for cell-specific RNA labeling. This novel metabolic labeling method can be employed to identify RNAs that come from a cell or interest (i.e., human iPSC lines). When RNAs in such cells are labeled, they can be isolated and purified. Described herein demonstrates that when animals are pulse-labeled with 5-vinyluridine it is incorporated into brain RNA, and is dependent on transplantation of mutant UPRT-expressing iPS-derived human microglia.

5-vinyl-uracil (Alfa Aesar #4437903) was prepared in sterile conditions at 4 times the concentration in 100% DMSO, then diluted to 25% DMSO by adding corn oil to a final concentration of 500 mM. The mixture was heated to 37° C. for 30-60 until homogenous. Mice are weighed before intraperitoneal injection (IP) injection to ensure close to 150 mg/kg are injected per treatment. For an (˜20 g) adult mouse typically 60-70 μl are IP injected per treatment. Mice are monitored for signs of distress. After 24-48 hours of treatment, mice were sacrificed and brains were immediately placed into 2 ml of RNAlater and left in 4° C. for 3-5 days. After removing brains from RNAlater, they were homogenized with RNAse-free 1.4 mm steel beads (Next Advance SSB14B-RNA) for 60 seconds and centrifuged at 4° C. for 5 min, maximum speed to separate the fatty layer. The bottom layer is collected, homogenized with 200 ul chloroform and centrifuged for 20 minutes, at 4° C. The aqueous layer is collected and 500 ul isopropanol added. After centrifugation for 30 minutes 4° C., the RNA pellet is washed in 70% Ethanol twice. After removing ethanol, the RNA pellet is resuspended in nuclease-free water and nanodrop to determine the concentration. From each sample, 15 μg of RNA was subjected in tetrazine-biotin ligation at 37° C., 500 RPM for 3 hours. The biotinylated RNA was purified with RNA Zymo Clean and Concentrator #5 and eluted with nuclease-free water. 2 μg of purified RNA was spotted onto a N+ Hybond membrane and UV-crosslinked at 254 nm (120,000 uJ). Membrane is blocked in blocking buffer (0.12 M NaCl, 0.016 M Na2HPO4, 0.008 M NaH2PO4, 0.17 M SDS) and followed by incubation with high sensitivity streptavidin-HRP (Fisher Scientific, Cat #: P121130) at 1:5000 dilution in blocking buffer for 5 minutes. The membrane was washed twice in a Wash A buffer (1:10 dilution of blocking buffer) and twice in Wash B buffer (Tris-saline buffer). It was then incubated for 1-5 min. in ECL Chemiluminescent Substrate (Fisher Scientific, Cat #: P132106) and imaged on a ChemiDoc MP imaging system (Bio-Rad).

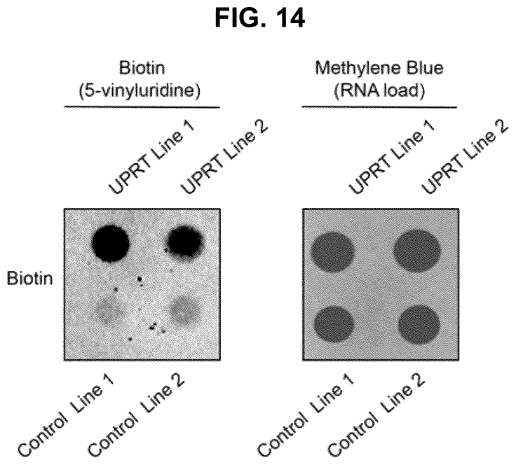

After total brain isolation, RNA was isolated using trizol RNA extraction. Extracted RNA was biotinylated using “click” chemistry. The presence of biotin was characterized using high sensitivity streptavidin-HRP. The expectation from this experiment is that UPRT(+)-microglia brains would have evidence for RNA incorporation by biotin dot blot. As shown in FIG. 14, consistent with that prediction only isolated RNA from UPRT(+)-microglia brains have observable signals. In the UPRT(−)-microglia brains a significant biotin signal was not observed as measured by dot blot (FIG. 14). Overall, these results demonstrate that the presently engineered UPRT enzyme, when paired with UPRT in transplanted cells is labeled cell-specific RNA. This cell-specific metabolic labeling is therefore consistent with the goal of using novel chemical methods to identify cell-specific RNAs that could be deployed for biomarker analyses.

Although there has been shown and described the preferred embodiment of the present invention, it will be readily apparent to those skilled in the art that modifications may be made thereto which do not exceed the scope of the appended claims. Therefore, the scope of the invention is only to be limited by the following claims. In some embodiments, the figures presented in this patent application are drawn to scale, including the angles, ratios of dimensions, etc. In some embodiments, the figures are representative only and the claims are not limited by the dimensions of the figures. In some embodiments, descriptions of the inventions described herein using the phrase “comprising” includes embodiments that could be described as “consisting essentially of” or “consisting of”, and as such the written description requirement for claiming one or more embodiments of the present invention using the phrase “consisting essentially of” or “consisting of” is met.

What is claimed is:

1. A method comprising:
a) xenotransplanting genetically modified differentiated cells into an area of interest in an animal model;
b) treating the animal model with an RNA analog;
c) extracting total RNA from the area of interest in the animal model;
d) producing a fraction of the RNA extracted in (c) by:
i) attaching a label to the RNA comprising the RNA analog; and
ii) isolating the labeled RNA from the total RNA extracted from the area of interest in the animal model; and
e) analyzing the RNA isolated from (d).

2. The method of claim 1, where in genetically modified differentiated cells comprise a genetically modified Uracil PhosphoRibosylTransferase (UPRT) enzyme

3. A method comprising:
a) xenotransplanting genetically modified differentiated cells into an area of interest in an animal model;
wherein the differentiated cells comprise a genetically modified Uracil PhosphoRibosylTransferase (UPRT) enzyme;
b) treating the animal model with an RNA analog;
c) extracting total RNA from the area of interest in the animal model;
d) producing a fraction of the RNA extracted in (c) by:
i) attaching a label to the RNA comprising the RNA analog; and
ii) isolating the labeled RNA from the total RNA extracted from the area of interest in the animal model; and
e) analyzing the RNA isolated from (d).

4. The method of any of claims 1-3, wherein the RNA analog comprises uracil-based analogs.

5. The method of claim 4, wherein the uracil-based analogs comprise 5-ethynylpyrimidine-2,4(1H,3H)-dione (5EU), 5-Vinylpyrimidine-2,4(1H,3H)-dione (5VU), 5-azido methyl uracil (5mAzU), 5-Azidopyrimidine-2,4(1H,3H)-dione (5AzU), or a combination thereof.

6. A method comprising:
a) xenotransplanting genetically modified differentiated cells into an area of interest in an animal model;
wherein the differentiated cells comprise a genetically modified Uracil PhosphoRibosylTransferase (UPRT) enzyme;
b) treating the animal model with an uracil analog;
c) extracting total RNA from the area of interest in the animal model;
d) producing a fraction of the RNA extracted in (c) by:
i) attaching a label to the RNA comprising the uracil analog; and
ii) isolating the labeled RNA from the total RNA extracted from the area of interest in the animal model; and
e) analyzing the RNA isolated from (d).

7. The method of any of claim 1-6, further comprising extracting total RNA brain, plasma, Cerebrospinal fluid (CSF), urine, or a combination thereof from the animal model.

8. The method of claim 6, wherein the uracil analog comprise 5-ethynylpyrimidine-2,4(1H,3H)-dione (5EU), 5-Vinylpyrimidine-2,4(1H,3H)-dione (5VU), 5-azido methyl uracil (5mAzU), 5-Azidopyrimidine-2,4(1K3H)-dione (5AzU), or a combination thereof.

9. The method of any of claim 1-7, wherein the genetically modified UPRT enzyme is a genetically modified Toxoplasma gondii Uracil PhosphoRiboxylTransferase (TgUPRT) enzyme.

10. The method of any of claim 1-9, wherein genetically modified UPRT enzyme comprises point mutations.

11. The method of any of claims 1-10, wherein the point mutations comprise M166A, A168G, Y228A, or a combination thereof.

12. A method comprising:
a) xenotransplanting genetically modified differentiated cells into an area of interest in an animal model;
b) treating the animal model with an amino acid analog;
c) extracting total protein from the area of interest in the animal model;
d) producing a fraction of the protein extracted in (c) by:
i) attaching a label to the protein comprising the amino acid analog; and
ii) isolating the labeled protein from the total protein extracted from the area of interest in the animal model; and
e) analyzing the protein isolated from (d).

13. The method of claim 12, where in genetically modified differentiated cells comprise a genetically modified Methionine tRNA Synthase (MetRS)

14. A method comprising:
a) xenotransplanting genetically modified differentiated cells into an area of interest in an animal model;
wherein the differentiated cells comprise a genetically modified Methionine tRNA Synthase (MetRS) enzyme.
b) treating the animal model with an amino acid analog;
c) extracting total protein from the area of interest in the animal model;
d) producing a fraction of the protein extracted in (c) by:
i) attaching a label to the protein comprising the amino acid analog; and
ii) isolating the labeled protein from the total protein extracted from the area of interest in the animal model; and
e) analyzing the protein isolated from (d).

15. The method of any of claim 12-14, further comprising extracting total protein from brain, plasma, Cerebrospinal fluid (CSF), urine, or a combination thereof from the animal model.

16. The method of any of claims 12-15, wherein the amino acid analog comprises azidonorleucine (ANL), azidotyrosine, and/or azidophenylalanine

17. The methods of any of claim 12-16, wherein the MetRS enzyme comprises a point mutation.

18. The method of claim 17, wherein the point mutation comprises a L272G (MethL272G).

19. A method comprising:
a) xenotransplanting genetically modified differentiated cells into an area of interest in an animal model;
b) treating the animal of the model with an RNA and/or an amino acid analog;
c) extracting total RNA and/or total protein from the area of interest in the animal model;
d) producing a fraction of the RNA and/or the protein extracted in (c) by:
i) attaching a label to the RNA and/or the protein comprising the RNA analog and/or the amino acid analog; and
ii) isolating the labeled RNA and/or the labeled protein from the total RNA and/or the total protein extracted from the area of interest in the animal model; and
e) analyzing the RNA and/or protein isolated from (d)

20. The method of claim 19, further comprising extracting total RNA from a brain, plasma, Cerebrospinal fluid (CSF), urine, or a combination thereof from the animal model.

21. The method of claim 19, further comprising extracting total protein from a brain, plasma, Cerebrospinal fluid (CSF), urine, or a combination thereof from the animal model.

22. The method of claim 19, wherein the genetically modified differentiated cells comprise a genetically modified Uracil PhosphoRibosylTransferase (UPRT) enzyme, a genetically modified Methionine tRNA Synthase (MetRS) enzyme, or a combination thereof.

23. The method of claim 22, wherein the genetically modified UPRT enzyme is a genetically modified Toxoplasma gondii Uracil PhosphoRiboxylTransferase (TgUPRT) enzyme.

24. The method of claim 23, wherein genetically modified UPRT enzyme comprises point mutations.

25. The method of claim 24, wherein the point mutations comprise M166A, A168G, Y228A, or a combination thereof.

26. The method of claim 22, wherein the genetically modified MetRS enzyme comprises a point mutation.

27. The method of claim 26 wherein the point mutation comprises a L272G (MethL272G).

28. The method of claim 19, wherein the RNA analog comprises uracil-based analogs.

29. The method of claim 28, wherein the uracil-based analogs comprise 5-ethynylpyrimidine-2,4(1H,3H)-dione (5EU), 5-Vinylpyrimidine-2,4(1H,3H)-dione (5VU), 5-azido methyl uracil (5mAzU), 5-Azidopyrimidine-2,4(1H,3H)-dione (5AzU), or a combination thereof.

30. The method of claim 19, wherein the amino acid analog comprises azidonorleucine (ANL), azidotyrosine, and/or azidophenylalanine.

31. The method of any of claims 1-30, further comprising differentiating stem cells.

32. The method of claim 31, wherein the stem cells are selected from a group consisting of embryonic (ESCs), induced pluripotent stem cells (iPSCs), neuronal stem cells, or derivatives thereof

33. The method of any of claims 1-32, wherein the differentiated cells are human differentiated cells.

34. The method of any of claim 1-33, wherein the differentiated cells are selected from a group consisting of neurons, microglia, astrocytes, oligodendrocytes and other cells of the central nervous system.

35. The method of any of claims 1-34, wherein the area of interest is the nervous system.

36. The method of any of claims 1-34, wherein the area of interest is the brain.

37. The method of any of claims 1-34, wherein the area of interest is the spinal cord.

38. The method of any of claims 1-37, wherein the animal model is a chimeric animal model.

39. The method of any of claims 1-37, wherein the animal model is an animal model of disease.

40. The method of claim 39, wherein the animal model of disease is an animal model of Alzheimer's Disease.

41. The method of claim 40, wherein the animal model of Alzheimer's Disease comprises beta-amyloid plaques.

42. The method of claim 41, wherein the fraction of labeled RNA changes in response to the beta-amyloid plaques.

43. The method of claim 40, wherein the animal model of Alzheimer's Disease comprises neurofibrillary tangles.

44. The method of claim 43, wherein the fraction of labeled RNA changes in response to the neurofibrillary tangles.

45. The method of claim 39, wherein the animal model of disease is an animal model of Parkinson's Disease.

46. The method of claim 39, wherein the animal model of disease is an animal model of frontotemporal dementia

47. The method of any of claims 1-46, wherein the animal model is an animal model of injury.

48. The method of claim 47, wherein the animal model of injury is a spinal cord injury animal model.

49. The method of claim 47, wherein the animal model of injury is a traumatic brain injury animal model.

50. The method of claims 1-49, wherein the animal model is a control animal model.

51. The method of claim 50, wherein the control animal model has no disease and/or injury.

52. The method of any of claim 1-51, wherein the method is for biomarker discovery and comprises comparing labeled RNA and/or labeled protein isolated from total RNA and/or total protein extracted from an animal model of disease to labeled RNA and/or labeled protein isolated from total RNA and/or total protein extracted from a control animal

53. The method of any of claim 1-52, wherein the method is for biomarker discovery and comprises comparing labeled RNA and/or labeled protein isolated from total RNA and/or total protein extracted from an animal model of injury to labeled RNA and/or labeled protein isolated from total RNA and/or total protein extracted from a control animal model.

54. The method of any of claims 1-53, wherein the method is for cell-specific RNA labeling and/or cell-specific protein labeling.

55. The method of any of claims 1-54, wherein the method provides cell-specific biorthogonal metabolic labeling.

56. A method comprising:
a) xenotransplanting human pluripotent stem cell derived microglia (iMGL) into an animal model's brain;
wherein the iMGL comprise a genetically modified Uracil PhosphoRiboxylTransferase (UPRT) enzyme;
b) treating the animal of the model with an uracil analog;
c) extracting total RNA from the animal model's brain;
d) producing a fraction of the RNA extracted in (c) by:
i) attaching a label to the RNA comprising the uracil analog; and
ii) isolating the labeled RNA from the total RNA extracted from the brain;
e) analyzing the RNA isolated from (d).

57. The method of claim 56, further comprising extracting total RNA brain, plasma, Cerebrospinal fluid (CSF), urine, or a combination thereof from the animal model.

58. The method of claim 56, wherein the uracil analog comprise 5-ethynylpyrimidine-2,4(1H,3H)-dione (5EU), 5-Vinylpyrimidine-2,4(1H,3H)-dione (5VU), 5-azido methyl uracil (5mAzU), 5-Azidopyrimidine-2.4(1H,3H)-dione (5AzU), or a combination thereof.

59. The method of claim 56, wherein the genetically modified UPRT enzyme is a genetically modified Toxoplasma gondii Uracil PhosphoRiboxylTransferase (TgUPRT) enzyme.

60. The method of claim 59, wherein genetically modified UPRT enzyme comprises point mutations.

61. The method of claim 60, wherein the point mutations comprise M166A, A168G, Y228A, or a combination thereof.
